(19) United States
(12) Patent Application Publication
(10) Pub. No.: US 2004/0132730 A1
(43) Pub. Date: Jul. 8, 2004

(54) Inhibitors of TGFbeta

(76) Inventors: Jonathan Axon, Milpitas, CA (US); Sarvajit Chakravarty, Mountain View, CA (US); Sundeep Dugar, San Jose, CA (US); Glenn McEnroe, San Mateo, CA (US); Alison Murphy, Milpitas, CA (US)

Correspondence Address: MORRISON & FOERSTER LLP, 3811 VALLEY CENTRE DRIVE, SUITE 500, SAN DIEGO, CA, 92130-2332, US

(21) Appl. No.: 10/660,115

(22) Filed: Sep. 10, 2003

Related U.S. Application Data
(60) Provisional application No. 60/409,870, filed on Sep. 10, 2002.

(51) Int. Cl. A61K 31/53; A61K 31/513; C 07D 4 3/02

(52) U.S. Cl. 514/241; 514/269; 514/275; 544/209; 544/319; 544/328

(57) ABSTRACT

Certain appropriately substituted forms of pyrimidine and triazine are useful in the treatment to conditions associated with enhanced TGFβ activity.

### RELATED APPLICATIONS

The application claims priority to U.S. Provisional Patent Application No. 60/409,870, filed Sep. 10, 2003, which is hereby incorporated by reference in its entirety.

### FIELD OF THE INVENTION

The invention relates to methods of treating various disorders associated with enhanced activity of transforming growth factor beta (TGFβ). More specifically, it concerns derivatives of pyrimidine and triazine that are useful in these methods.

### BACKGROUND ART

Transforming growth factor-beta (TGFβ) denotes a superfamily of proteins that includes, for example, TGFβ1, TGFβ2, and TGFβ3, which are pleiotropic modulators of cell growth and differentiation, embryonic and bone development, extracellular matrix formation, hematopoiesis, immune and inflammatory responses (Roberts and Sporn Handbook of Experimental Pharmacology (1990) 95:419-58; Massague, et al., Ann. Rev. Cell. Biol. (1990) 6:597-646). Other members of this superfamily include activin, inhibin, bone morphogenic protein, and Mullerian inhibiting substance. The members of the TGFβ family initiate intracellular signaling pathways leading ultimately to the expression of genes that regulate the cell cycle, control proliferative responses, or relate to extracellular matrix proteins that mediate outside-in cell signaling, cell adhesion, migration and intercellular communication.

Therefore, inhibitors of the TGFβ intracellular signaling pathway are useful treatments for fibroproliferative diseases. Specifically, fibroproliferative diseases include kidney disorders associated with unregulated TGFβ activity and excessive fibrosis including glomerulonephritis (GN), such as mesangial proliferative GN, immune GN, and crescentic GN. Other renal conditions include diabetic nephropathy, renal interstitial fibrosis, renal fibrosis in transplant patients receiving cyclosporin, and HIV-associated nephropathy. Collagen vascular disorders include progressive systemic sclerosis, polymyositis, scleroderma, dermatomyositis, eosinophilic fascitis, morphea, or those associated with the occurrence of Raynaud's syndrome. Lung fibroses resulting from excessive TGFβ activity include adult respiratory distress syndrome, COPD, idiopathic pulmonary fibrosis, and interstitial pulmonary fibrosis often associated with autoimmune disorders, such as systemic lupus erythematosus and scleroderma, chemical contact, or allergies. Another autoimmune disorder associated with fibroproliferative characteristics is rheumatoid arthritis.

Fibroproliferative conditions can be associated with surgical eye procedures. Such procedures include retinal reattachment surgery accompanying proliferative vitreoretinopathy, cataract extraction with intraocular lens implantation, and post glaucoma drainage surgery.

The compounds of the invention herein are derivatives of pyrimidine or triazine. PCT publication WO01/47921 describes pyrimidine and triazine compounds that are inhibitors of kinase activities associated with various inflammatory conditions, as opposed to the treatment of fibroproliferative disorders described herein. The above mentioned PCT publication describes the use of the compounds disclosed only for treatment of the inflammatory aspects of certain autoimmune diseases. Further, the compounds described differ from those described herein by virtue of the substitutions required on the pyrimidine or triazine nucleus; among other distinctions, the compounds disclosed in this publication do not include phenyl bound directly to the pyrimidine or triazine ring.

### DISCLOSURE OF THE INVENTION

The invention is directed to methods and compounds useful in treating conditions that are characterized by TGFβ activity. These conditions are, most prominently, fibroproliferative diseases.

The compounds of the invention have been found to inhibit TGFβ and are thus useful in treating diseases mediated by the activity of this family of factors. The compounds of the invention are of the formula

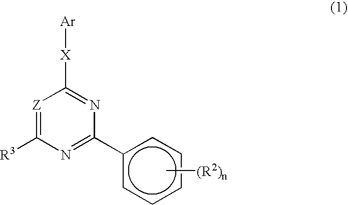

and the pharmaceutically acceptable salts and prodrug forms thereof; wherein

Ar represents an optionally substituted aromatic or optionally substituted heteroaromatic moiety containing 5-12 ring members wherein said heteroaromatic moiety contains one or more O, S, and/or N with a proviso that the optionally substituted Ar is not

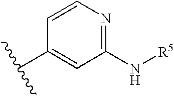

wherein R⁵ is H, alkyl (1-6C), alkenyl (2-6C), alkynyl (2-6C), an aromatic or heteroaromatic moiety containing 5-11 ring members;

X is NR¹, O, or S;

R¹ is H, alkyl (1-8C), alkenyl (2-8C), or alkynyl (2-8C);

Z represents N or CR⁴;

each of R³ and R⁴ is independently H, or a non-interfering substituent;

each R² is independently a non-interfering substituent; and

n is 0, 1, 2, 3, 4, or 5. In one embodiment, if n>2, and the R²'s are adjacent, they can be joined together to form a 5 to 7 membered non-aromatic, heteroaromatic, or aromatic ring containing 1 to 3 heteroatoms where each heteroatom can independently be O, N, or S.

In preferred embodiments, Ar represents an optionally substituted aromatic or optionally substituted heteroaromatic moiety containing 5-9 ring members wherein said heteroaromatic moiety contains one or more N; or

R¹ is H, alkyl (1-8C), alkenyl (2-8C), or alkynyl (2-8C); or

Z represents N or CR⁴; wherein

R⁴ is H, alkyl (1-10C), alkenyl (2-10C), or alkynyl (2-10C), acyl (1-10C), aryl, alkylaryl, aroyl, O-aryl, O-alkylaryl, O-aroyl, NR-aryl, NR-alkylaryl, NR-aroyl, or the hetero forms of any of the foregoing, halo, OR, NR₂, SR, —SOR, —NRSOR, —NRSO₂R, —SO₂R, —OCOR, —NRCOR, —NRCONR₂, —NRCOOR, —OCONR₂, —COOR, —SO₃R, —CONR₂, —SO₂NR₂, —CN, —CF₃, or —NO₂, wherein each R is independently H or alkyl (1-10C) or a halo or heteroatom-containing form of. said alkyl, each of which may optionally be substituted. Preferably R⁴ is H, alkyl (1-10C), OR, SR or NR₂ wherein R is H or alkyl (1-10C) or is O-aryl; or

R³ is defined in the same manner as R⁴ and preferred forms are similar, but R³ is independently embodied; or

each R² is independently alkyl (1-8C), alkenyl (2-8C), alkynyl (2-8C), acyl (1-8C), aryl alkylaryl, aroyl, O-aryl, O-alkylaryl, O-aroyl, NR-aryl, NR-alkylaryl, NR-aroyl, or the hetero forms of any of the foregoing, halo, OR, NR₂, SR, —SOR, —NRSOR, —NRSO₂R, —NRSO₂R₂, —SO₂R, —OCOR, —OSO₃R, —NRCOR, —NRCONR₂, —NRCOOR, —OCONR₂, —COOR, —SO₃R, —CONR₂, SO₂NR₂, —CN, —CF₃, or —NO₂, wherein each R is independently H or lower alkyl (1-4C). Preferably R² is halo, alkyl (1-6C), OR, SR or NR₂ wherein R is H or lower alkyl (1-4C), more preferably halo; or

n is 0-3.

The optional substituents on the aromatic or heteroaromatic moiety represented by Ar include alkyl (1-10C), alkenyl (2-10C), alkynyl (2-10C), acyl (1-10C), aryl, alkylaryl, aroyl, O-aryl, O-alkylaryl, O-aroyl, NR-aryl, NR-alkylaryl, NR-aroyl, or the hetero forms of any of the foregoing, halo, OR, NR₂, SR, —SOR, —NRSOR, —NRSO₂R, —SO₂R, —OCOR, —NRCOR, —NRCONR₂, —NRCOOR, —OCONR₂, —COOR, —SO₃R, —CONR₂, —SO₂NR₂, —CN, —CF₃, and/or NO₂, wherein each R is independently H or lower alkyl (1-4C). Preferred substituents include alkyl, OR, NR₂, O-alkylaryl and NH-alkylaryl.

In general, any alkyl, alkenyl, alkynyl, acyl, or aryl group contained in a substituent may itself optionally be substituted by additional substituents. The nature of these substituents is similar to those recited with regard to the primary substituents themselves.

The invention is also directed to pharmaceutical compositions containing one or more compounds of formula (1) or their pharmaceutically acceptable salts or prodrug forms thereof, as active ingredients and to methods of treating fibroproliferative conditions using these compounds and compositions.

### MODES OF CARRYING OUT THE INVENTION

The compounds of formula (1) are useful in treating conditions which are characterized by overactivity of TGFβ. Conditions “characterized by enhanced TGFβ activity” include those wherein TGFβ synthesis is stimulated so that TGFβ is present in enhanced amount or wherein TGFβ latent protein is undesirably activated or converted to active TGFβ protein or wherein TGFβ receptors are upregulated or wherein the TGFβ protein shows enhanced binding to cells or extracellular matrix in the location of the disease. Thus, in either case, “enhanced activity” refers to any condition wherein the effectiveness of TGFβ is undesirably high, regardless of the cause.

As used herein, “TGFβ” refers to the superfamily which includes TGFβ1, TGFβ2, and TGFβ3 as well as other members of the family known or which became known in the art such as inhibin, bone morphogenic protein, and the like. One or more of these family members may be elevated in the conditions which the compounds of the invention are designed to ameliorate or prevent.

The Invention Compounds

The compounds useful in the invention are derivatives of pyrimidine or triazine containing mandatory substituents at positions corresponding to the 2- and 4-positions of pyrimidine. In general, a pyrimidine nucleus is preferred, although triazine nucleus is also within the scope of the invention as illustrated below. Further non-interfering substituents may also be included.

As used herein, a “non-interfering substituent” is a substituent which leaves the ability of the compound of formula (1) to inhibit TGFβ activity qualitatively intact. Thus, the substituent may alter the degree of inhibition, but as long as the compound of formula (1) retains the ability to inhibit TGFβ activity, the substituent will be classified as “noninterfering.”

As used herein, the term “alkyl,” “alkenyl” and “alkynyl” include straight-chain, branched-chain and cyclic monovalent substituents, containing only C+H when they are unsubstituted. Examples include methyl, ethyl, isobutyl, cyclohexyl, cyclopentylethyl, 2-propenyl, 3-butynyl, and the like. Typically, the alkyl, alkenyl and alkynyl substituents contain 1-10C (alkyl) or 2-10C (alkenyl or alkynyl). Preferably they contain 1-6C (alkyl) or 2-6C (alkenyl or alkynyl).

Heteroalkyl, heteroalkenyl and heteroalkynyl are similarly defined but may contain 1-3O, S or N heteroatoms or combinations thereof within the backbone residue.

As used herein, “acyl” encompasses the definitions of alkyl, alkenyl, alkynyl, and heteroacyl includes the related heteroforms, each of which are coupled to an additional residue through a carbonyl group.

“Aromatic” moiety or “aryl” moiety refers to a monocyclic or fused bicyclic moiety such as phenyl or naphthyl; “heteroaromatic” also refers to monocyclic or fused bicyclic ring systems containing one or more heteroatoms selected from O, S and N. The inclusion of a heteroatom permits inclusion of 5-membered rings as well as 6-membered rings. Thus, typical aromatic/heteroaromatic systems include pyridyl, pyrimidyl, indolyl, benzimidazolyl, benzotriazolyl, isoquinolyl, quinolyl, benzothiazolyl, benzofuranyl, thienyl, furyl, pyrrolyl, thiazolyl, oxazolyl, imidazolyl and the like. Because tautomers are theoretically possible, phthalimido is also considered aromatic, and phthalimido-substituted alkyl and phthalimido-substituted alkoxy are preferred embodiments of R³ and R⁴. Any monocyclic or fused ring bicyclic system which has the characteristics of aromaticity in terms of electron distribution throughout the ring system is included in this definition. Typically, the ring systems contain 5-12 ring member atoms.

Similarly, “arylalkyl” and “heteroarylalkyl” refer to aromatic and heteroaromatic systems which are coupled to another residue through a carbon chain, including substituted or unsubstituted, saturated or unsaturated, carbon chains, typically of 1-8C, or the hetero forms thereof. These carbon chains may also include a carbonyl group, thus making them able to provide substituents as an acyl or heteroacyl moiety.

In general, any alkyl, alkenyl, alkynyl, acyl, or aryl group contained in a substituent may itself optionally be substituted by additional substituents. The nature of these substituents is similar to those recited with regard to the primary substituents themselves. Thus, where an embodiment of, for example, R⁴ is alkyl, this alkyl may optionally be substituted by the remaining substituents listed as embodiments for R⁴ where this makes chemical sense, and where this does not undermine the size limit of alkyl per se; e.g., alkyl substituted by alkyl or by alkenyl would simply extend the upper limit of carbon atoms for these embodiments. However, alkyl substituted by aryl, amino, alkoxy, and the like would be included within the scope of the invention. The features of the invention compounds are defined by formula (1) and the nature of the substituents is less important as long as the substituents do not interfere with the stated biological activity of this basic structure.

Non-interfering substituents embodied by R², R³ and R⁴, include, but are not limited to, alkyl, alkenyl, alkynyl, halo, OR, NR₂, SR, —SOR, —SO₂R, —OCOR, —NRCOR, —NRCONR₂, —NRCOOR, —OCONR₂, —RCO, —COOR, SO₂R, NRSOR, NRSO₂R, —SO₃R, —CONR₂, SO₂NR₂, wherein each R is independently H or alkyl (1-8C), —CN, —CF₃, and NO₂, and like substituents. R³ and R⁴ can also be H. Preferred embodiments for R³ and R⁴ are H, alkyl (1-10C) or a heteroatom-containing form thereof, each optionally substituted, especially (1-4C) alkyl; alkoxy (1-8C), acylamido, aryloxy, arylalkyloxy, especially wherein the aryl group is a phthalimido group, and alkyl or arylalkyl amine. Preferred embodiments of R² include lower alkyl, alkoxy, and halo, preferably halo. Halo, as defined herein includes fluoro, chloro, bromo and iodo. Fluoro and chloro are preferred.

Preferably, R¹ is H or lower alkyl (1-4C), more preferably H.

Preferably Ar is optionally substituted phenyl, 2-, 3- or 4-pyridyl, indolyl, 2- or 4-pyrimidyl, pyridazinyl, benzotriazol or benzimidazolyl. More preferably Ar is phenyl, pyridyl, or pyrimidyl. Each of these embodiments may optionally be substituted with a group such as alkyl, alkenyl, alkynyl, aryl, O-aryl, O-alkylaryl, O-aroyl, NR-aryl, N-alkylaryl, NR-aroyl, halo, OR, NR₂, SR, —OOCR, —NROCR, RCO, —COOR, —CONR₂, and/or SO₂NR₂, wherein each R is independently H or alkyl (1-8C), and/or by —CN, —CF₃, and/or NO₂. Alkyl, alkenyl, alkynyl and aryl portions of these may be further substituted by similar substituents. However, an optionally substituted Ar is not

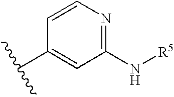

wherein R⁵ is H, alkyl (1-6C), alkenyl (2-6C), alkynyl (2-6C), an aromatic or heteroaromatic moiety containing 5-11 ring members wherein said heteroaromatic moiety contains one or more O, S, and/or N. Thus, when Ar is 4-pyridyl, the 2- or 6-position of the pyridyl is not a —NHR⁵ substituent.

Preferred substituents on Ar include alkyl, alkenyl, alkynyl, halo, OR, SR, NR₂ wherein R is H or alkyl (1-4C); and/or arylamino, arylalkylamino, including alkylamino which is substituted by more than one aryl. As stated above, any aryl or alkyl group included within a substituent may itself be substituted similarly. These substituents may occupy all available positions of the ring, preferably 1-2 positions, or more preferably only one position.

Any of the aryl moieties, including those depicted in formula (1) especially the phenyl moieties, may also comprise two substituents which, when taken together, form a 5-7 membered carbocyclic or heterocyclic aliphatic ring. Similarly, R⁴ may be bridged to R³ to obtain a 5-7 membered carbocyclic or heterocyclic ring.

The compounds of formula (1) may be supplied in the form of their pharmaceutically acceptable acid-addition salts including salts of inorganic acids such as hydrochloric, sulfuric, hydrobromic, or phosphoric acid or salts of organic acids such as acetic, tartaric, succinic, benzoic, salicylic, and the like. If a carboxyl moiety is present on the compound of formula (1), the compound may also be supplied as a salt with a pharmaceutically acceptable cation.

The compounds of formula (1) may also be supplied in the form of a “prodrug” which is designed to release the compound of formula (1) when administered to a subject. Prodrug formed designs are well known in the art, and depend on the substituents contained in the compound of formula (1). For example, a substituent containing sulfhydryl could be coupled to a carrier which renders the compound biologically inactive until removed by endogenous enzymes or, for example, by enzymes targeted to a particular receptor or location in the subject.

In the event that any of the substituents of formula (1) contain chiral centers, as some, indeed, do, the compounds of formula (1) include all stereoisomeric forms thereof, both as isolated stereoisomers and mixtures of these stereoisomeric forms.

Synthesis of the Invention Compounds

A number of synthetic routes may be employed to produce the compounds of the invention. In general, they may be synthesized using reactions known in the art. One useful method, especially with regard to embodiments which contain nitrile substitutions (which also, of course, can be hydrolyzed to the corresponding carboxylic acids or reduced to the amines) is shown in reaction Scheme 1, shown below. This scheme is illustrated in Example 4. As indicated, in this and alternative approaches, an intermediate wherein the pyrimidine ring is halogenated is obtained; the halide is then displaced by an aryl amine. In this first illustrative method, the pyrimidine ring is generated in the synthetic scheme, resulting in the compound formed in reactions labeled a. Compounds 1, 2, and 3 were made according to this scheme.

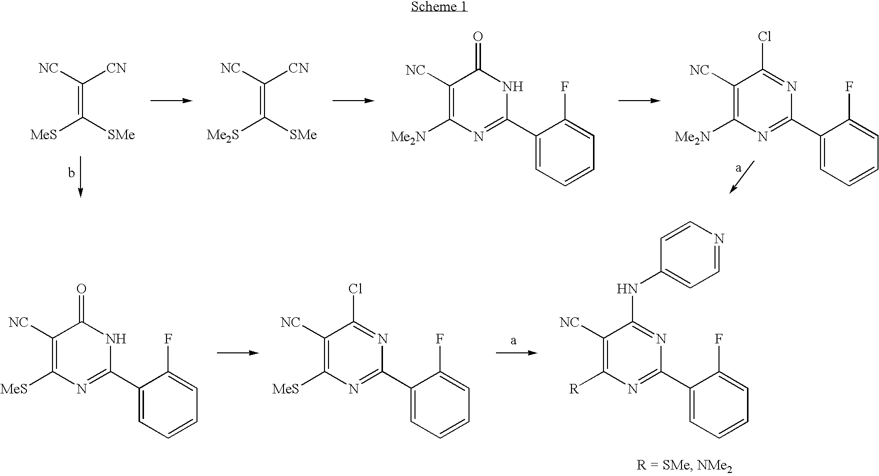

In reaction Scheme 2, which was used to prepare most of the illustrative compounds shown below, the pyrimidine ring is obtained by cyclizing an amido moiety and, again, a halo group on the pyrimidine ring is displaced by an aryl amide to obtain the compounds of the invention in step b. Further substitution on the resulting invention compound can then also be performed as shown in subsequent steps b¹, b², and b³. Compounds 9, 10, 12, 15, 17, 18, 19, 21-26, 31-40, 57, 58, 61, 64, 65, 67 and 69-73 in Table 1 were prepared according to this general scheme.

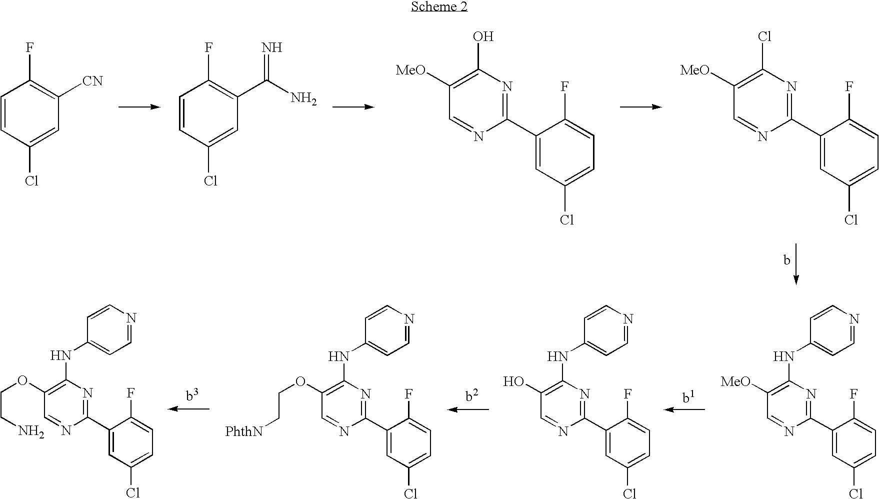

Reaction Schemes 3 and 4, shown below, provide alternative routes to the pyrimidine nucleus, and further substitution thereof. Compound 14 was prepared according to the general procedure outlined in scheme 3 and compounds 7 and 11 were prepared according to the general procedure outlined in scheme 4.

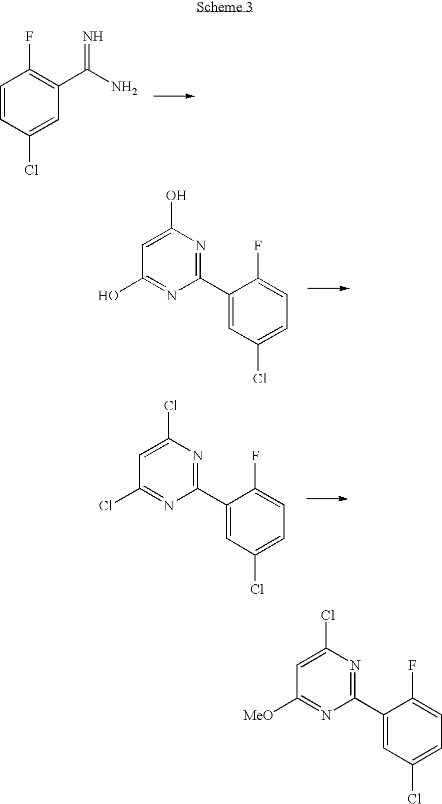

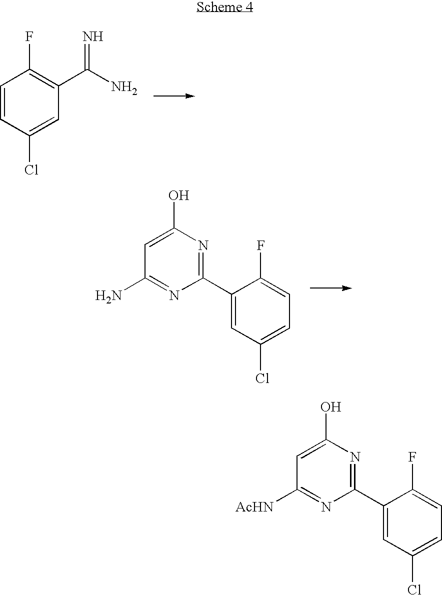

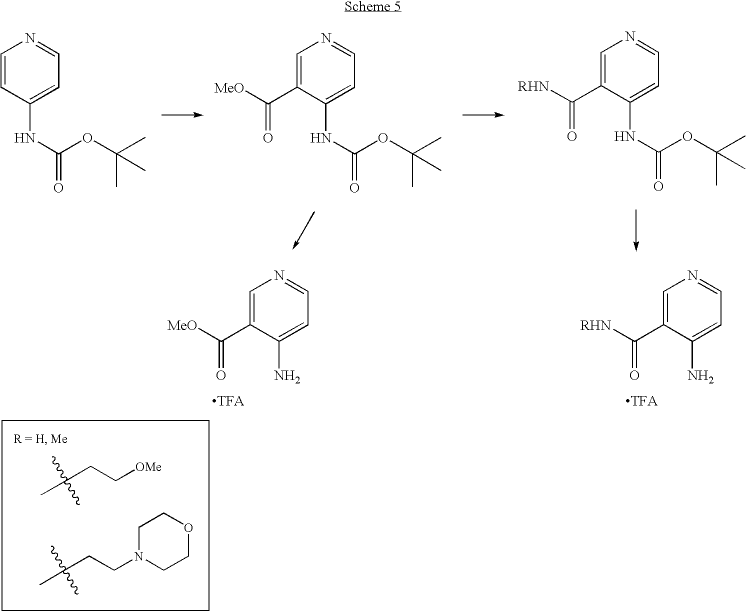

This scheme was generally used to synthesize compounds 61, 64, 69, 71 and 72 in Table 1.

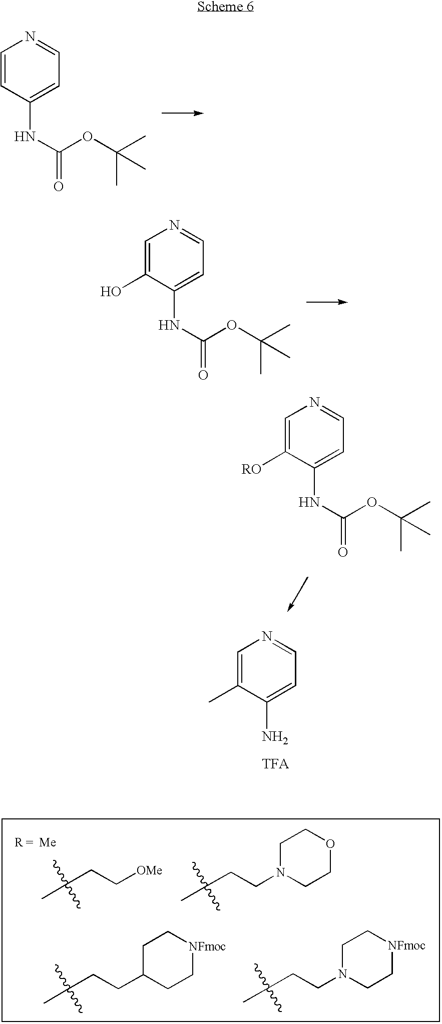

This scheme was generally used to synthesize compounds 18, 37, 38, 39, 67 and 73 in Table 1.

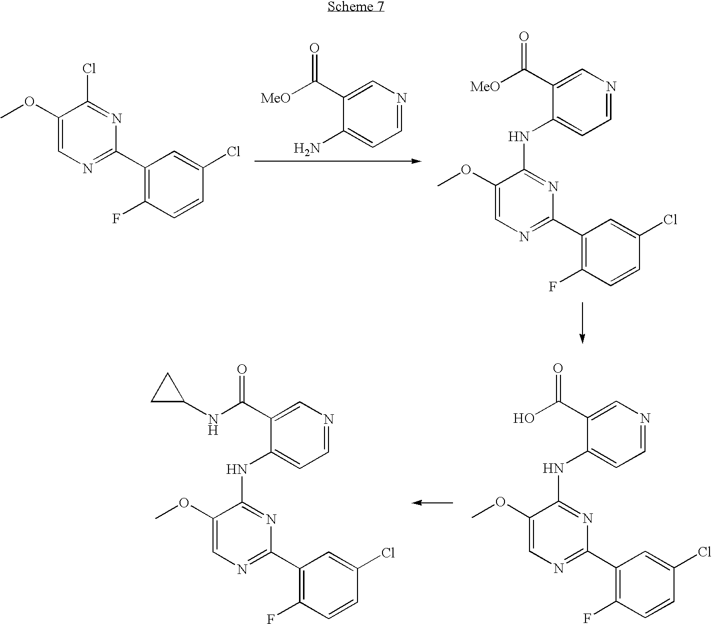

This scheme can be generally used to make methoxy pyrimidines. For example this scheme was and could be generally used to synthesize compounds 61, 64, 69, 71, 72, 74-81, 83 -106, 109, 111 and 112, in Table 1.

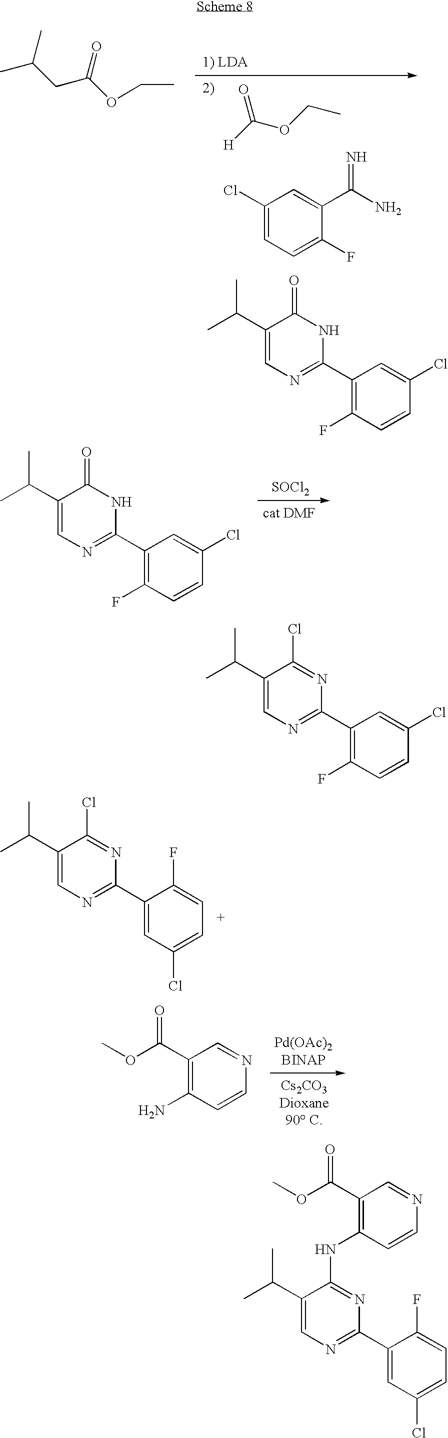

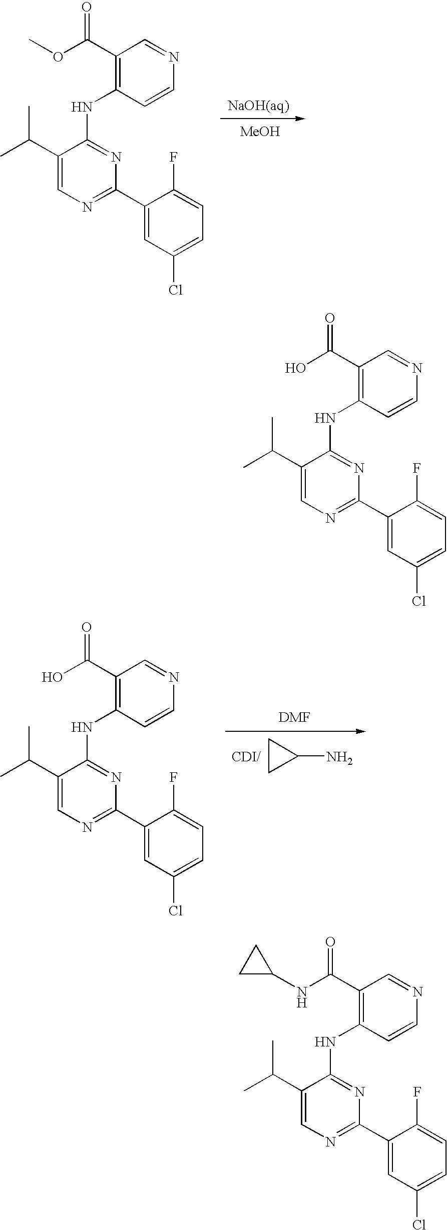

This scheme can be generally used to make isopropyl pyrimidines. This scheme was generally used to synthesize compounds 113, 115, 116, 121, 124-129, and 139 in Table 1.

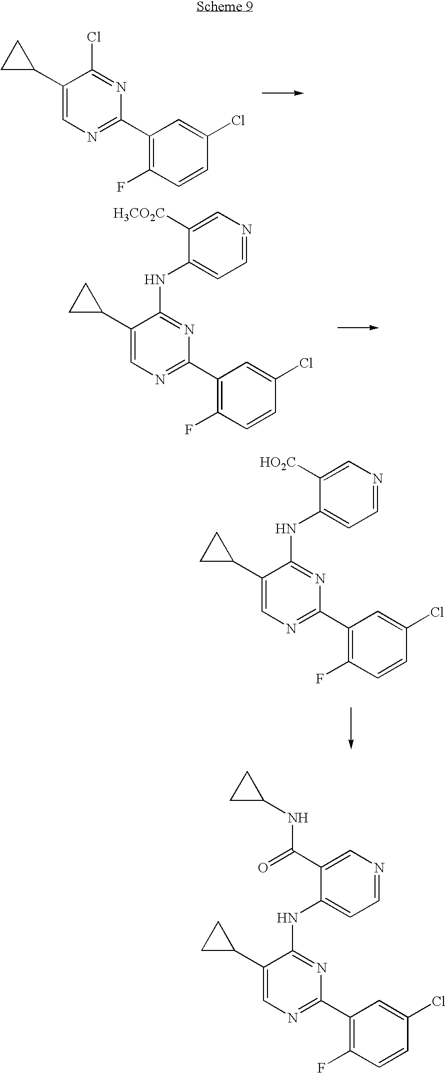

This scheme can be generally used to make cyclopropyl pyrimidines. For example, it was used to synthesize compounds 117-119, 122, and 130-134 in Table 1.

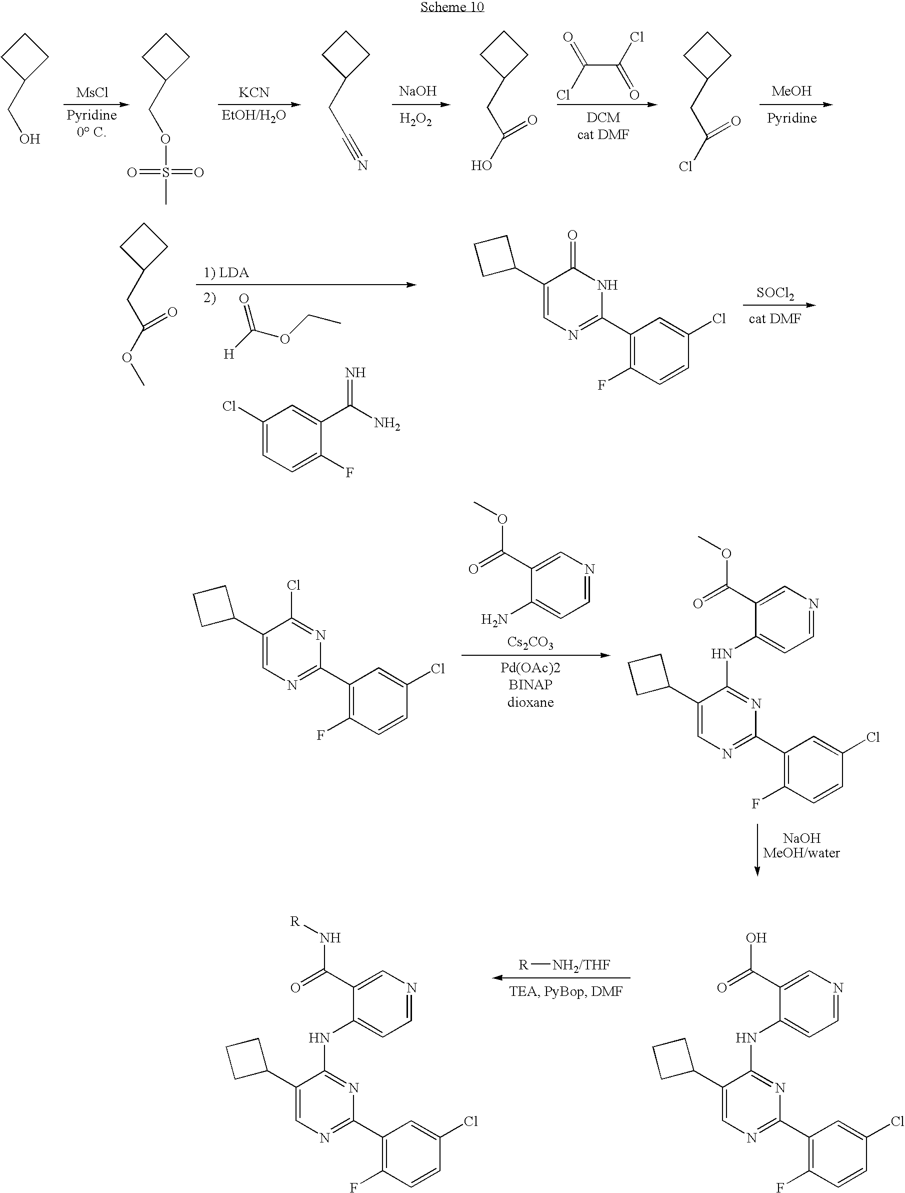

This scheme can be generally used to make cyclobutyl pyrimidines. For example, it was used to synthesize compounds 136-138 in Table 1.

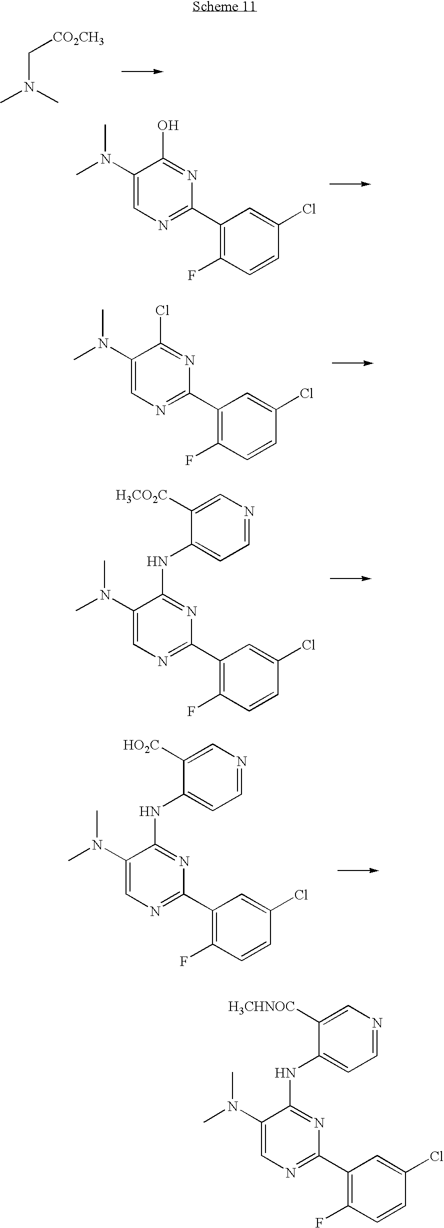

This scheme was generally used to synthesize compound 135 in Table 1.

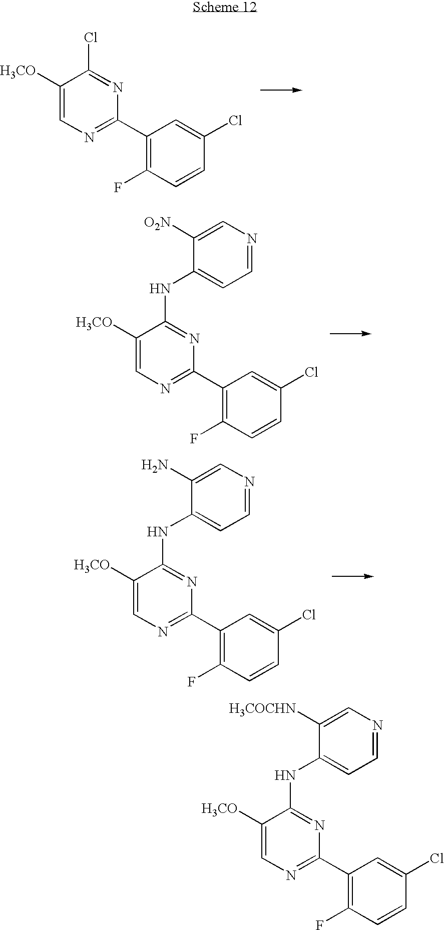

This scheme was generally used to synthesize compounds 107,108, and 110 in Table 1.

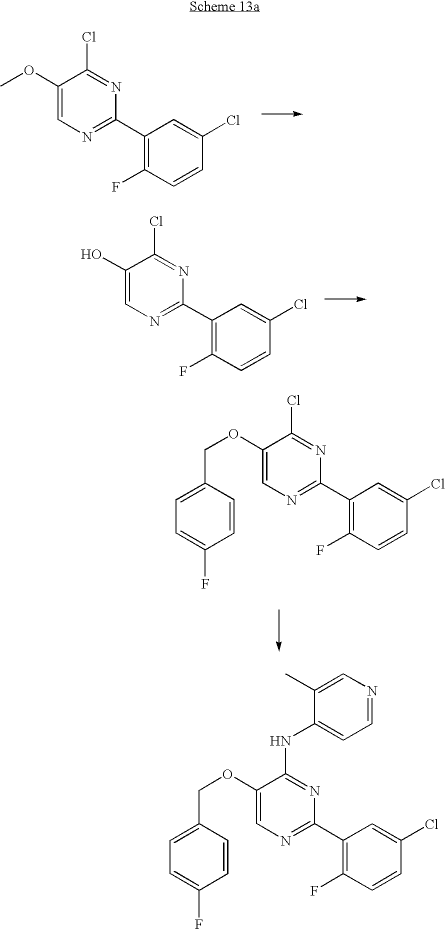

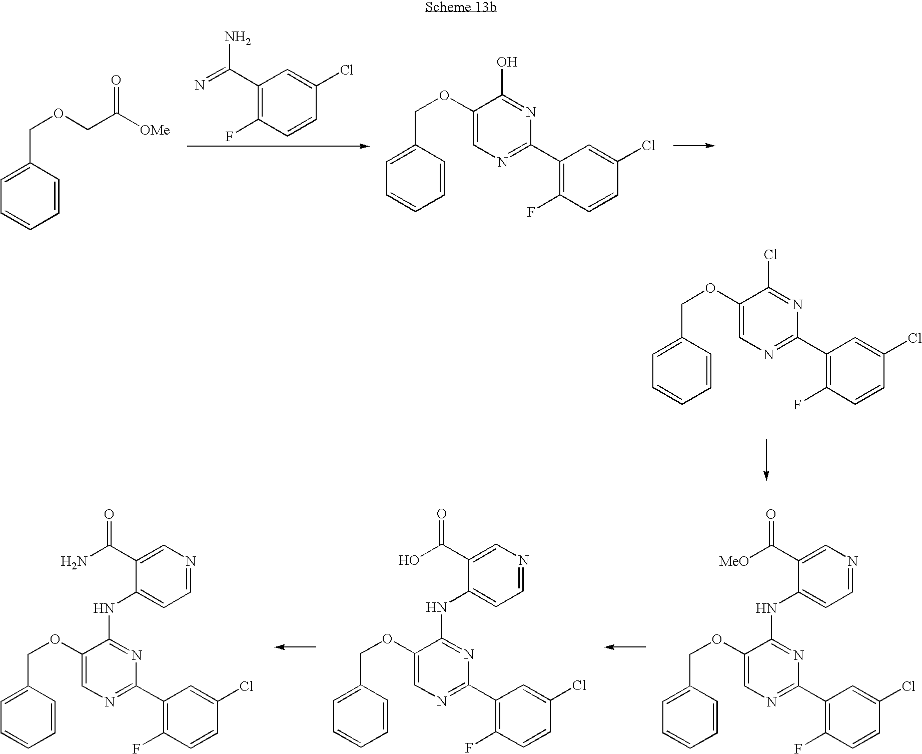

Schemes 13a and 13b can be generally used to make benzyloxy pyrimidines. For example, scheme 13a was used to synthesize compounds 148-152, 157 and 158. Scheme 13b was used to synthesize compounds 140-147 and 153-156.

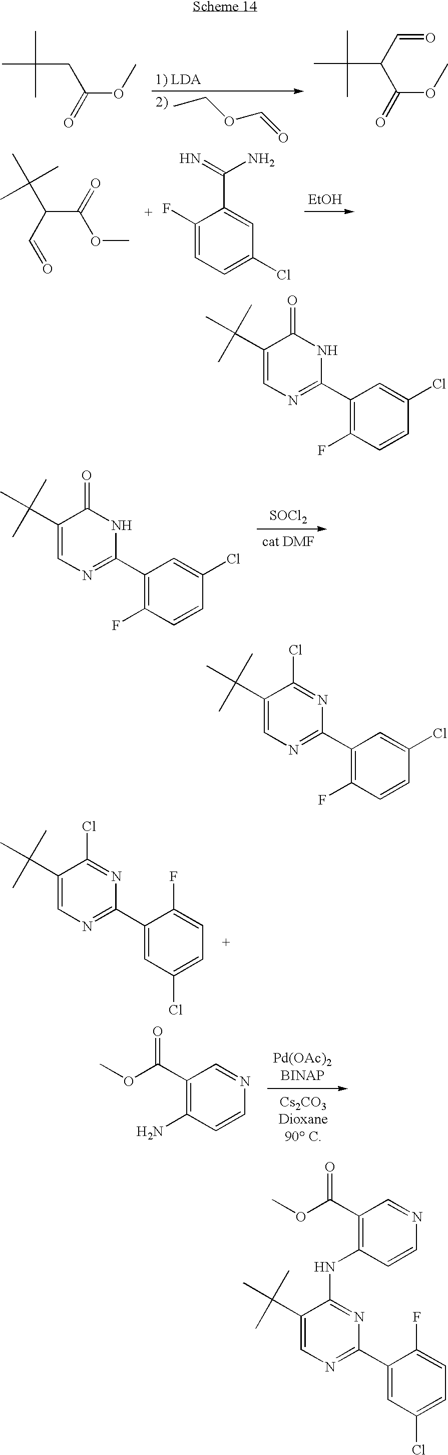

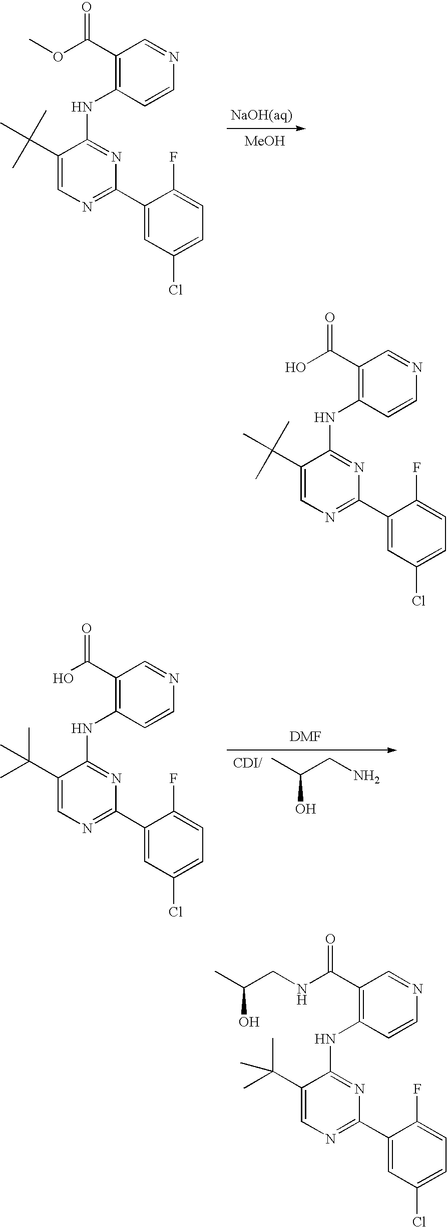

This scheme can be generally used to synthesize t-butyl pyrimidines. For example, this scheme was used to synthesize compounds 159 and 160 in Table 1

Administration and Use

The compounds of the invention are useful in treating conditions associated with fibroproliferation. Thus, the compounds of formula (1) or their pharmaceutically acceptable salts or prodrug forms are used in the manufacture of a medicament for prophylactic or therapeutic treatment of mammals, including humans, in respect of conditions characterized by excessive activity of TGFβ.

TGFβ inhibition activity is useful in treating fibroproliferative diseases, treating collagen vascular disorders, treating eye diseases associated with a fibroproliferative condition, venting excessive scarring, treating neurological conditions and other conditions that are targets for TGFβ inhibitors and in preventing excessive scarring that elicits and accompanies restenosis following coronary angioplasty, cardiac fibrosis occurring after infarction and progressive heart failure, and in hypertensive vasculopathy, and keloid formation or hypertrophic scars occurring during the healing of wounds including surgical wounds and traumatic lacerations.

Neurological conditions characterized by TGFβ production include CNS injury after traumatic and hypoxic insults, Alzheimer's disease, and Parkinson's disease.

Other conditions that are potential clinical targets for TGFβ inhibitors include myelofibrosis, tissue thickening resulting from radiation treatment, nasal polyposis, polyp surgery, liver cirrhosis, and osteoporosis.

Diseases benefited by TGFβ inhibition include cardiovascular diseases such as congestive heart failure, dilated cardiomyopathy, myocarditis, or vascular stenosis associated with atherosclerosis, angioplasty treatment, or surgical incisions or mechanical trauma; kidney diseases associated with fibrosis and/or sclerosis, including glomerulonephritis of all etiologies, diabetic nephropathy, and all causes of renal interstitial fibrosis, including hypertension, complications of drug exposure, such as cyclosporin, HIV-associated nephropathy, transplant nephropathy, chronic ureteral obstruction; hepatic diseases associated with excessive scarring and progressive sclerosis, including cirrhosis due to all etiologies, disorders of the biliary tree, and hepatic dysfunction attributable to infections such as hepatitis virus or parasites; syndromes associated with pulmonary fibrosis with consequential loss of gas exchange or ability to efficiently move air into and out of the lungs, including adult respiratory distress syndrome, idiopathic pulmonary fibrosis, or pulmonary fibrosis due to infectious or toxic agents such as smoke, chemicals, allergens, or autoimmune disease; all collagen vascular disorders of a chronic or persistent nature including progressive systemic sclerosis, polymyositis, scleroderma, dermatomyositis, fascists, or Raynaud's syndrome, or arthritic conditions such as rheumatoid arthritis; eye diseases associated with fibroproliferative states, including proliferative vitreoretinopathy of any etiology or fibrosis associated with ocular surgery such as retinal reattachment, cataract extraction, or drainage procedures of any kind; excessive or hypertrophic scar formation in the dermis occurring during wound healing resulting from trauma or surgical wounds; disorders of the gastrointestinal tract associated with chronic inflammation, such as Crohn's disease or ulcerative colitis or adhesion formation as a result of trauma or surgical wounds, polyposis or states post polyp surgery; chronic scarring of the peritoneum associated with endometriosis, ovarian disease, peritoneal dialysis, or surgical wounds; neurological conditions characterized by TGFβ production or enhanced sensitivity to TGFβ, including states post-traumatic or hypoxic injury, Alzheimer's disease, and Parkinson's disease; diseases of the joints involving scarring sufficient to impede mobility or produce pain, including states post-mechanical or surgical trauma, osteoarthritis and rheumatoid arthritis; and cancer.

The modulation of the immune and inflammation systems by TGFβ (Wahl, et al., Immunol. Today (1989) 10:258-61) includes stimulation of leukocyte recruitment, cytokine production, and lymphocyte effector function, and inhibition of T-cell subset proliferation, B-cell proliferation, antibody formation, and monocytic respiratory burst. TGFβ is a stimulator for the excess production of extracellular matrix proteins, including fibronectin and collagen. It also inhibits the production of enzymes that degrade these matrix proteins. The net effect is the accumulation of fibrous tissue which is the hallmark of fibroproliferative diseases.

TGFβ is active as a homodimer, but is synthesized and secreted from cells as an inactive latent complex of the mature homodimer and proregions, called latency associated protein (LAP). These proteins bind to each other through noncovalent interactions (Lyons and Moses, Eur. J. Biochem. (1990) 187:467). LAP is often disulfide-linked to separate gene products, called latent TGFβ binding proteins or LTBP's. These latent forms provide stability for the mature cytokine and a means for targeting it to the extracellular matrix and cell surfaces (Lawrence, Eur. Cytokine Network (1996) 7:363-74). Activation of the latent complex occurs after secretion from cells and is believed to result from the action of proteases, such as plasmin (Munger, et al., Kidney Intl. (1997) 51:1376-82), on LAP, thrombospondin-1 binding (Crawford, et al., Cell (1998) 93:1159-70), and binding to the integrin v6 (Munger, et al., Cell (1999) 319-28).

Other than αvβ there is a variety of cell surface proteins/receptors that transduce the signals initiated by binding of the active TGFβ ligand to its receptors. These include types I, II, III, IV, and V. Type IV is present only in the pituitary gland while the others are ubiquitous. The binding affinities among the three isoforms for the type I and II receptors differ such that these two receptors bind TGFβ1 and TGFβ3 more tightly than TGFβ2 (Massague, Cell (1992) 69:1067-70).

The type IV receptor or endoglin has a similar isoform binding profile in contrast to the type III receptor, betaglycan, which binds equally well to all three isoforms (Wang, et al., Cell (1991) 67:797-805; Lopez-Casillas, Cell (1991) 67:785-95). The type V receptor binds to IGFBP-3 and is thought to have an active kinase domain similar to the type I and II receptors. Cloning of the type I and type II receptors demonstrated the existence of cytoplasmic serine/threonine kinase domains (Wrana, et al., Cell (1992) 71:1003-14; Lin, et al., Cell (1992) 68:775-85; Ibid. 71:1069; Massague, Cell (1992) 69:1067-70). Initiation of the TGFβ signaling pathway results from the binding of the TGFβ ligand to the extracellular domain of the type II receptor (Massague, Ann. Rev. Biochem. (1998) 67:753-91). The bound receptor then recruits type I receptor into a multimeric membrane complex, whereupon the constitutively active type II receptor kinase phosphorylates and activates type I receptor kinase. The function of the type I receptor kinase is to phosphorylate a receptor-associated co-transcription factor, smad-2/3, thereby releasing it into the cytoplasm where it binds to smad-4. This smad complex translocates into the nucleus, associates with a DNA-binding cofactor, such as Fast-1, binds to enhancer regions of specific genes, and activates transcription. The expression of these genes leads to the synthesis of cell cycle regulators that control proliferative responses or extracellular matrix proteins that mediate outside-in cell signaling, cell adhesion, migration, and intercellular communication.

The manner of administration and formulation of the compounds useful in the invention and their related compounds will depend on the nature of the condition, the severity of the condition, the particular subject to be treated, and the judgment of the practitioner; formulation will depend on mode of administration. As the compounds of the invention are small molecules, they are conveniently administered by oral administration by compounding them with suitable pharmaceutical excipients so as to provide tablets, capsules, syrups, and the like. Suitable formulations for oral administration may also include minor components such as buffers, flavoring agents and the like. Typically, the amount of active ingredient in the formulations will be in the range of 5%-95% of the total formulation, but wide variation is permitted depending on the carrier. Suitable carriers include sucrose, pectin, magnesium stearate, lactose, peanut oil, olive oil, water, and the like.

The compounds useful in the invention may also be administered through suppositories or other transmucosal vehicles. Typically, such formulations will include excipients that facilitate the passage of the compound through the mucosa such as pharmaceutically acceptable detergents.

The compounds may also be administered topically, for topical conditions such as psoriasis, or in formulation intended to penetrate the skin. These include lotions, creams, ointments and the like which can be formulated by known methods.

The compounds may also be administered by injection, including intravenous, intramuscular, subcutaneous or intraperitoneal injection. Typical formulations for such use are liquid formulations in isotonic vehicles such as Hank's solution or Ringer's solution.

Alternative formulations include nasal sprays, liposomal formulations, slow-release formulations, and the like, as are known in the art.

Any suitable formulation may be used. A compendium of art-known formulations is found in Remington's Pharmaceutical Sciences, latest edition, Mack Publishing Company, Easton, Pa. Reference to this manual is routine in the art.

The dosages of the compounds of the invention will depend on a number of factors which will vary from patient to patient. However, it is believed that generally, the daily oral dosage will utilize 0.001-100 mg/kg total body weight, preferably from 0.01-50 mg/kg and more preferably about 0.01 mg/kg-10 mg/kg. The dose regimen will vary, however, depending on the conditions being treated and the judgment of the practitioner.

It should be noted that the compounds of formula (1) can be administered as individual active ingredients, or as mixtures of several embodiments of this formula. The compounds of the invention may be used as single therapeutic agents or in combination with other therapeutic agents. Drugs that could be usefully combined with these compounds include natural or synthetic corticosteroids, particularly prednisone and its derivatives, monoclonal antibodies targeting cells of the immune system, antibodies or soluble receptors or receptor fusion proteins targeting immune or non-immune cytokines, and small molecule inhibitors of cell division, protein synthesis, or mRNA transcription or translation, or inhibitors of immune cell differentiation or activation.

As indicated above, although the compounds of the invention may be used in humans, they are also available for veterinary use in treating animal subjects.

The following examples are intended to illustrate, but not to limit, the invention.

### EXAMPLE 1

### Synthesis of [2-(3-chlorophenyl)-pyrimidin-4-yl]pyridin-4-yl amine

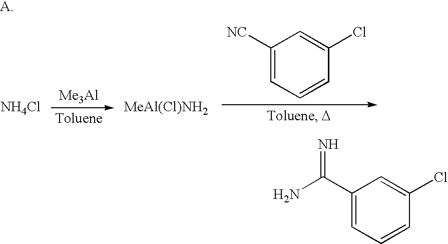

To a vigorously stirred, cooled (0° C.) suspension of (pestle-ground) ammonium chloride (1.17 g, 21.8 mmol) in dry toluene (7 mL) was added a solution of trimethylaluminum (10.9 mL, 2M solution in hexanes, 21.8 mmol) dropwise over 20 min. Effervescence occurred on addition. The mixture was stirred at r.t. for 15 min. To this solution was added a solution of 3-chlorobenzonitrile (1.0 g, 7.2 mmol) in dry toluene (5 mL) dropwise over 10 min. The solution was heated to 80° C. for 12 h then cooled and transferred slowly into a vigorously stirred slurry of silica gel (30 g) in chloroform (100 mL). The slurry was left stirred at r.t. for 10 min., then filtered. The filter cake was washed with methanol (3×100 mL) and the filtrate evaporated to a white solid that was dissolved in 10% aq. HCl (100 mL) and diethyl ether (50 mL). The solution was shaken and the organic layer discarded. The aqueous layer was basified to pH 14 with satd. aq. NaOH, and extracted with chloroform (3×100 mL). The organic extracts were dried over sodium sulfate and evaporated to a yellow oil that solidified (813 mg, 72%). EIMS: 154 M+.

Alternatively, an analogous intermediate can be synthesized using Lithium bis(trimethylsilyl)amide:

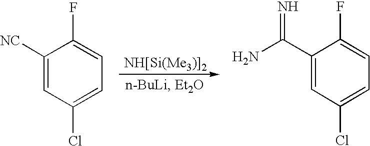

To a stirred 0° C. solution of 1,1,1,3,3,3-Hexamethyldisilazane (63 mL, 0.3 mmol) in dry diethyl ether was added dropwise n-Butyl lithium (2M in hexanes, 119 mL, 0.3 mmol). A white suspension formed, to which was added 2-Fluoro-5-chlorobenzonitrile (21.0 g, 0.14 mmol) over 5 min. The resultant orange mixture was allowed to warm to r.t. and stirred for 2 h. The mixture was cooled to 0° C. and the reaction quenched by the addition of 3M HCl (aq.) (240 mL). The mixture was stirred for 0.5 h before water (600 mL) was added. The purple organic layer was discarded and the aqueous layer basified to pH 14 with satd. NaOH (aq.). The aqueous layer was extracted with CHCl₃ (5×100 mL) and the organic extracts dried over Na₂SO₄. Evaporation yielded the desired product as a yellow solid (16.2 g, 73% yield).

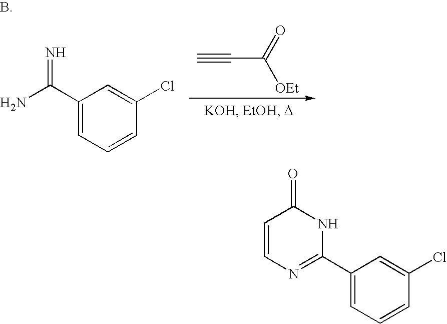

To a solution of 3-Chlorobenzamidine (1 g, 6.47 mmol) in dry ethanol (20 mL) was added ethyl propiolate (983 mL, 9.70 mmol) dropwise over 1 min. The solution was heated to 60° C. and a solution of potassium hydroxide (640 mg, 9.70 mmol) in dry ethanol (15 mL) was added dropwise over 1 h. Once added, the mixture was heated at 80° C. for 24 h, then cooled and evaporated. The residue was dissolved in water and the solution acidified with 10% aq. HCl to pH 4, whereupon a white precipitate formed, which was filtered and dried in vacuo (742 mg, 56%).

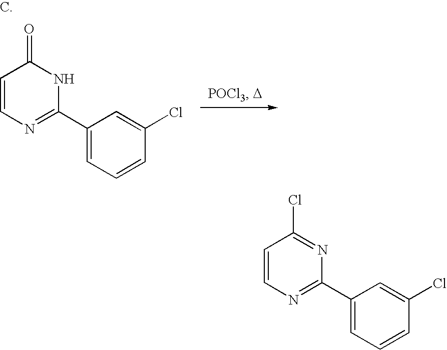

A suspension of the crude 2-(3-Chlorophenyl)-pyrimidin-4-one (197 mg, 0.9 mmol) in phosphorus oxychloride (5 mL) was heated to reflux for 0.5 h, then cooled and evaporated. The residue was purified by chromatography (CHCl₃) to yield the desired product as a white solid (191 mg, 89% yield). EIMS : 225 M+.

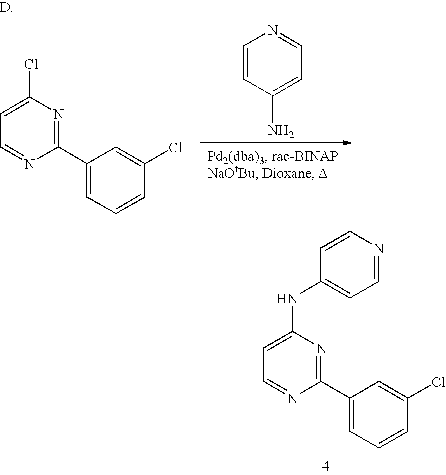

To a stirred solution of chloropyrimidine (20 mg, 88.9, μmol) in dry dioxane (1 mL) stirred at r.t. under nitrogen was added Pd₂(dba)₃ (4 mg, 4.4 μmol), then rac-BINAP (4 mg, 6.6 μmol). To this purple solution was added dropwise a solution of 4-aminopyridine (28 mg, 0.293 mmol) in dry dioxane (1 mL), followed by sodium tert-butoxide (29 mg, 0.293 mmol). The brown mixture was heated to 80° C. for 12 h then cooled and filtered through a plug of celite with methanol as eluent. The filtrate was evaporated and the residue purified by preparative HPLC to yield the product as the trifluoroacetate salt (4.7 mg, 17%). ESMS: 282 M+.

### EXAMPLE 2

The following method was used for the preparation of compound 31 and is generally applicable to the synthesis of compounds 9, 10, 12, 15, 17, 18, 19, 21-26, 32-40, 57, 58, 61, 64, 65, 67, and 69-73 in Table 1.

Demethylation of Methyl Ether

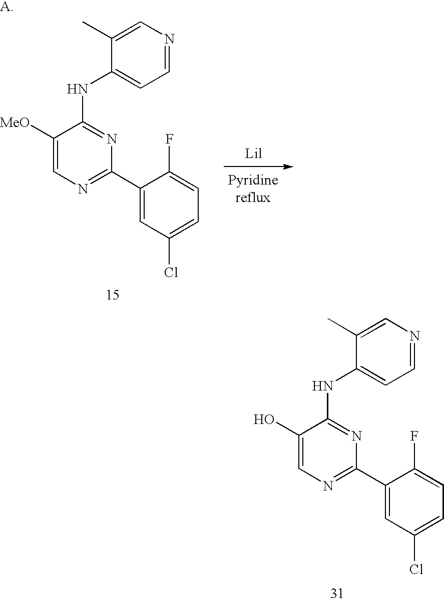

To a stirred r.t. solution of 2-(2-Fluoro-5-chlorophenyl)-4-(340 -methyl-4-aminopyridin-4-yl)-5-methoxypyrimidine (596 mg, 1.73 mmol) in dry pyridine (50 mL) was added lithium iodide (4 g). The mixture was heated to 130° C. for a total of 3 days. The mixture was evaporated and the residue purified by preparative HPLC to yield the product as the trifluoroacetate salt, a cream solid (400 mg, 70% yield). ESMS : 331 MH+.

Alkylation of Hydroxyl Group

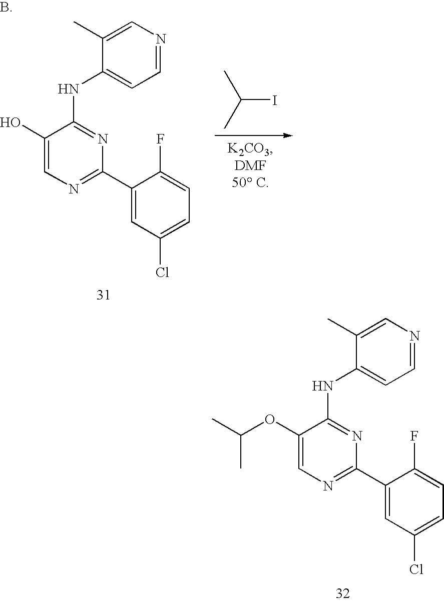

To a stirred r.t. solution of 2-(2-Fluoro-5-chlorophenyl)-4-(3′-methyl-4-aminopyridin-4-yl)-5-hydroxypyrimidine (50 mg, 0.15 mmol) in dry DMF (5 mL) was added finely ground K₂CO₃ (42 mg, 0.30 mmol) followed by 2-iodopropane (31 mg, 0.18 mmol). The solution was heated to 50° C. for 12 h, then evaporated. The residue was purified by radial chromatography (5% MeOH in CHCl₃) to yield the free base, which was converted by HCl(g)/Et₂O to the HCl salt. Lyophilization yielded the desired product as a white solid (34 mg, 55% yield). ESMS : 373 MH+.

### EXAMPLE 3

### Synthesis of Cyano Substituted Compound

The following method was used for the synthesis of compounds 1, 2, and 3.

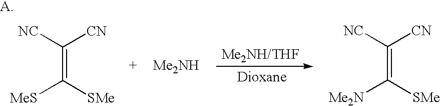

To a stirred r.t. solution of 2-[di(methylthio)methylidene]malononitrile (1 g, 5.9 mmol) in dry dioxane was added a solution of dimethylamine in THF (2M, 3.5 mL, 7.0 mmol). The mixture was left stirred at r.t. for 12 h and then evaporated to a yellow oil that solidified (1.01 g crude). The material was sufficiently pure for use in the next reaction. EIMS: 167 M+.

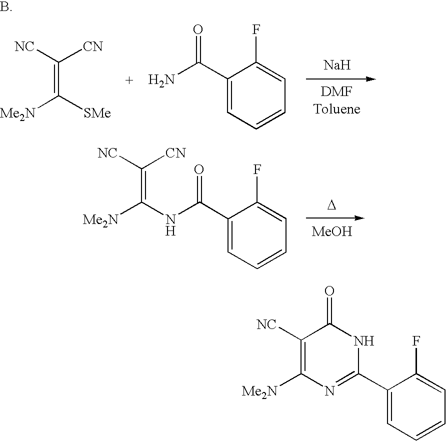

To a stirred r.t. solution of crude 2-[dimethylamino(methylthio)methylidene]malononitrile in dry DMF (30 mL) and dry toluene (30 mL) was added 2-Fluorobenzamide (817 mg, 5.9 mmol). Sodium hydride (60% suspension in mineral oil, 470 mg, 11.7 mmol) was added portionwise. The mixture was stirred at r.t. for 12 h then poured into iced water and acidified to pH 4-5 with 1M HCl (aq). The two layers were separated and the aqueous layer extracted with EtOAc (3×50 mL). The combined organic extracts were washed with brine then dried over MgSO₄. Evaporation yielded an orange oil that was dissolved in dry methanol (60 mL) and heated to reflux for 12 h then evaporated. The resultant orangesolid was purified by chromatography (1:1 CHCl₃: EtOAc) to yield the pure product as a pale yellow solid (629 mg, 42% over 3 steps). EIMS : 261 M+.

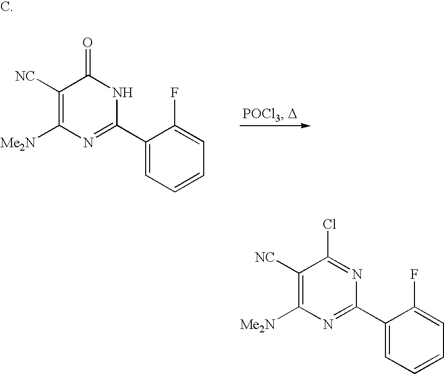

To a suspension of 2-(2-Fluorophenyl)-5-cyano-6-dimethylamino-pyrimidin-4-one (500 mg, 1.9 mmol) in phosphorus oxychloride (20 mL) was added N,N-dimethylaniline (235 mg, 1.9 mmol). The solid dissolved and the solution was heated to reflux for 4 h then cooled and evaporated. The brown residue was purified by chromatography (1:1 CHCl₃: EtOAc) to yield the desired product as a cream solid (226 mg, 43%). EIMS : 276 M+.

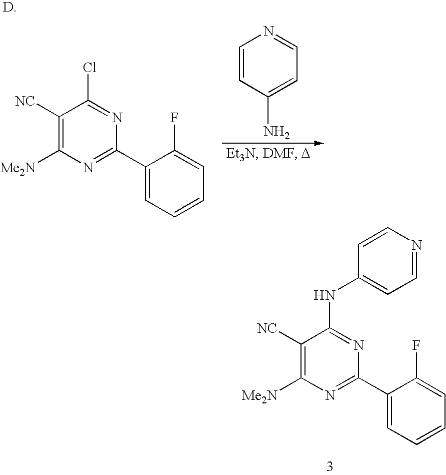

To a stirred r.t. solution of 2-(2-Fluorophenyl)-4-chloro-5-cyano-6-dimethylaminopyrimidine (223 mg, 0.8 mmol) in dry DMF (4 mL) was added 4-aminopyridine (152 mg, 1.6 mmol) and triethylamine (82 mg, 0.8 mmol). The solution was heated to reflux for 12 h then cooled and evaporated. The residue was shaken in CHCl₃ (30 mL) and 1N NaOH (aq) (30 mL). The layers were separated and the aqueous layer extracted with further CHCl₃ (3×30 mL). The combined organic extracts were dried over MgSO₄ and evaporated to a brown oily solid. Purification by chromatography (10% MeOH in CHCl₃) gave the desired free base, which was converted by HCl/Et₂O to the HCl salt, a white solid (46 mg, 17% yield). EIMS: 334 M+.

### EXAMPLE 4

### Synthesis of Compound 48

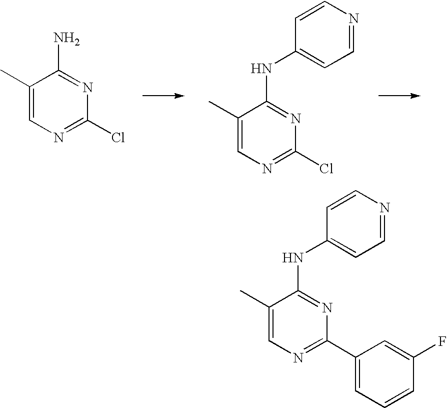

An oven dried sealed tube was charged with dioxane (10 mL), 4-Bromopyridine hydrochloride (4.7 g, 24.3 mmol), 2-Chloro-4-Amino-5-Methylpyrimidine (2.0 g, 16.2 mmol, Toronto Research), sodium tert-butoxide (4.6 g, 48.6 mmol), BINAP (760 mg, 1.21 mmol), and palladium(II) acetate (181 mg, 0.81 mmol) under nitrogen. The tube was placed into a 90° C. oil bath and heated for 18 h. The reaction mixture was then allowed to cool to room temperature, diluted with dichloromethane (10 mL), filtered through Celite, and concentrated. The crude product was purified by silica gel column chromatography (5% MeOH-DCM) to give 500 mg (15%) of the desired product. LCMS: 221 MH+.

An oven dried sealed tube was charged with acetonitrile (3 mL), water (1 mL) 2-chloro-4-(4-pyridylamino)-pyrimidine (46 mg, 0.20 mmol), 3-fluorophenylboronic acid (85 mg, 0.612 mmol), potassium carbonate (112 mg, 0.81 mmol) and Pd(PPh₃)₄ (14 mg, 0.02 mmol) under nitrogen. The tube was placed into a 90° C. oil bath and heated for 18 h. The reaction mixture was then allowed to cool to room temperature, diluted with ethyl acetate (10 mL), filtered through Celite, and concentrated. The crude product was purified by preparative TLC (3% MeOH-DCM) to give 25 mg (52%) of the desired product. LCMS: 281 MH+.

### EXAMPLE 5

### Synthesis of compound 63

This method is generally applicable to the synthesis of compounds 62, 63, 66 and 68 of Table 1.

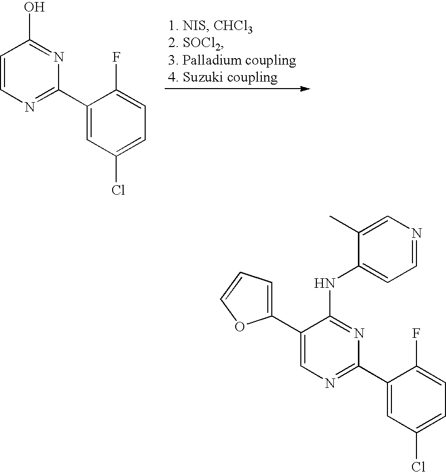

To a solution of pyrimidinone (3.65 g, 16 mmol), in dry chloroform was added NIS (5.5 g, 24 mmol) in one portion and the reaction mixture was heated to 60° C. overnight. The reaction mixture was cooled to r.t. and partitioned between chloroform and water. The organic layers were combined, washed with, brine, dried over MgSO₄, filtered and concentrated in vacuo and the residue purified by flash column chromatography to give the desired product (4.82 g, 84% yield) as a cream colored solid. ESMS : 350 (M+).

A suspension of the pyrimidine (2 g, 5.71 mmol) in SOCl₂ (5 ml) containing 2 drops of DMF was stirred under reflux for 5 h. The solution was then cooled to room temperature and concentrated under reduced pressure to give a solid that was dissolved in dry methylene chloride. The solution was cooled to 0° C. and ice was added followed by sat. NaHCO₃. The organic layer was separated, washed with brine, dried (MgSO₄), filtered and evaporated in vacuo to provide a crude white solid that was not further purified. ESMS: 368 (M+).

To a suspension of the imino chloride (500 mg, 1.42 mmol) in dioxane (5 ml) was added Pd₂(dba)₃ (65 mg, 0.07 mmol) followed by BINAP (66 mg, 0.11 mmol), 4-amino-3-picoline (230 mg, 2.13 mmol) and NaO^{t}Bu (273 mg, 2.84 mmol). The reaction mixture was heated to 90° C. for 15 h. The reaction mixture was cooled to r.t. and filtered through Celite and the crude material purified by flash column chromatography to give (152 mg, 24% yield) as a cream colored solid. ESMS: 440 (M+).

### EXAMPLE 6

### Preparation of Compound 80

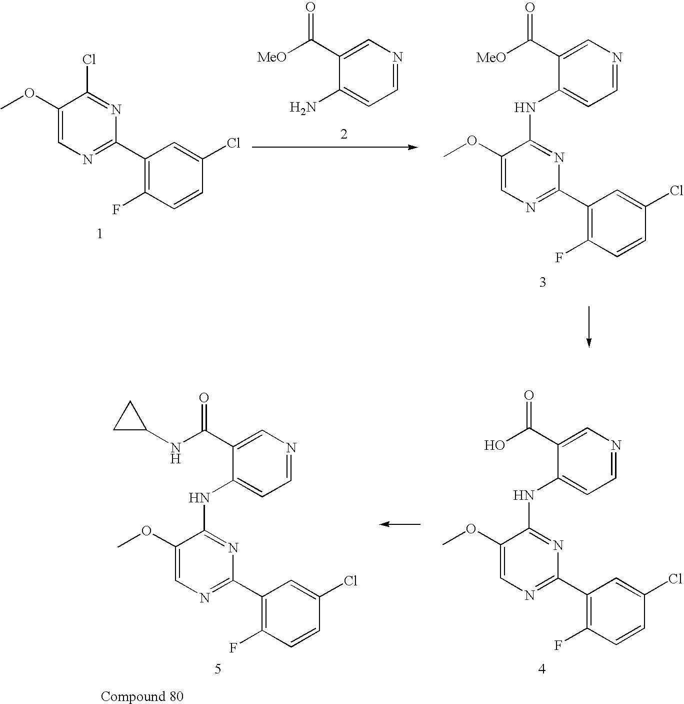

Preparation of 3:

The imino chloro compound 1 (5 g, 18.3 mmol, 1 eq), Pd2(dba)3 (670 mg, 0.7 mmol, 0.04 eq) and BINAP (684 mg, 1.1 mmol, 0.06 eq) were suspended in dioxane (280 mL) under N2. A solution/suspension of the amine 2 (3.07 g, 20.2 mmol, 1.1 eq) in dioxane (90 mL) was added at a moderate speed, followed by Cs2CO3 (11.9 g, 36.5 mmol, 2 eq). The mixture was then heated to 95° C. under N2 for 18 hours. The warm reaction mixture was then filtered through Celite and the Celite pad was washed with ethyl acetate (100 mL). The filtrate was then concentrated in vacuo to approx 100 mL in volume (not to dryness). The suspension was filtered and the solid washed with ethyl acetate and dried in vacuo. Product 3 was obtained as a cream solid 4.92 g, 69% yield: pure.

Preparation of 4:

A suspension of the Ester 3 (1.6 g, 4.1 mmol), NaOH (1.5-1.8 eq, 0.3 g, 7.5 mmol), water (5 mL) and dioxane (50 mL) was heated to 65° C. for 0.5 hour. The reaction was cooled to room temperature and 1M HCl solution was added until a pH 4 was obtained. The suspension was filtered and washed with water. The product 4 was dried in vacuo at 40° C. overnight., 1.1 g, 71% yield (cream solid)

Preparation of 5 (Compound 80):

A suspension of the acid 4 (1 g, 2.67 mmol) and CDI (0.865 g, 5.33 mmol, 2.0 eq) in dry DMF (20 mL) was heated at 75° C. for 0.5-2 hrs under N2. The reaction was cooled to room temperature and cyclopropylamine (0.3 mL, 4.1 mmol, 1.5 eq) and triethylamine (0.4 mL, 2.67 mmol) were added. The reaction was stirred for 18 hours. The reaction mixture was then filtered and the solid washed with ethyl acetate. The pure product was obtained as a white solid, 0.71 g, 65% yield.

### EXAMPLE 7

### Preparation of Compound 123

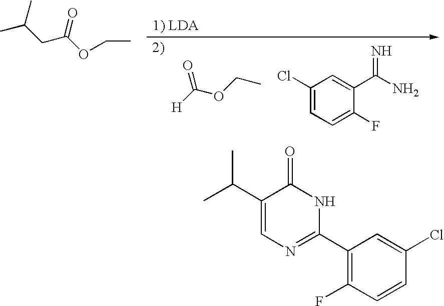

To a solution of disopropylamine (15.4 ml, 110 mmole) in 30 ml tetrahydofuran (anh.) at −20° C. was added dropwise, n-butyllithium (2.5M hexane, 48 ml, 120 mmole). The solution was stirred at 0° C. for 40 min. The mixture was then cooled to −78° C. and ethyl isovalerate (13.0 g, 100 mmole) was added dropwise, the reaction mixture was stirred at −78° C. for 30 min. Ethyl formate (7.41 g, 100 mmole) was then added and the reaction mixture was warmed to room temperature with stirring for 1 hour. 5-chloro-2-fluorobenzamidine (17.0 g, 100 mmole) was dissolved in tetrahydrofuran (40 ml) and added to the reaction mixture over 10 min, followed by refluxing for 18 hr. Removed solvent under vacuum and residue was suspended in chloroform (150 ml) and water (150 ml). The basic aqueous phase was separated and filtered to remove some precipitate. The filtrate was acidified with glacial acetic acid to pH 5 and extracted with ethyl acetate (2×250 ml), washed combined extracts with saturated sodium chloride, dried over sodium sulfate (anh.) and removed the solvent to give 3.43 g product.

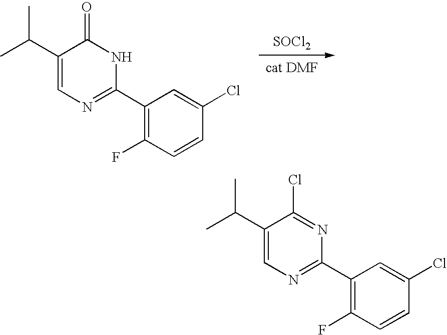

2-(5-chloro-2-fluorophenyl)-5-isopropylprimidine-4-one (3.43 g, 12.86 mmole) was suspended in thionyl chloride (15 ml, 205 mmole) and 3 drops DMF were added. The mixture was heated to 80° C. for 30 min, removed excess thionyl chloride under vacuum. The residue was treated with ice (50 ml) and chloroform (50 ml). Extracted product into chlororom layer. Washed chloroform with 10% sodium carbonate (cold) and dried chloroform layer over sodium sulfate (anh,) Removed solvent to give 3.32 g product.

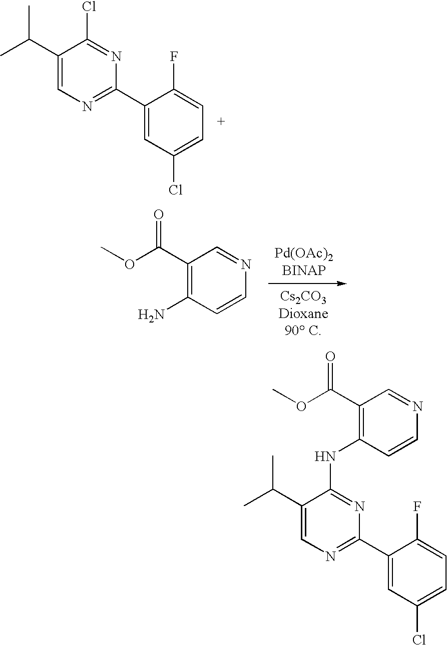

BINAP (233 mg, 0.375 mmole) and palladium(II)acetate (56.1 mg, 0.25 mmole were combined in 8 ml dioxane (anh) and heated for 5 min, followed by addition of 2-(5-chloro-2-fluorophenyl)-4-chloro-5-isoprpylpyrimidine (1.42 g, 5 mmole), methyl 4-amino-3-pyridinecarboxylate (912 mg, 6 mmole) and cesium carbonate (2.28 g, 7.0 mmole). The mixture was heated to 90° C. overnight. Removed dioxane under vacuum, the solid residue was tritureated with ethyl acetate (20 ml) and filtered to give 767 mg product which contains cesium carbonate and was used directly in next step without further purification.

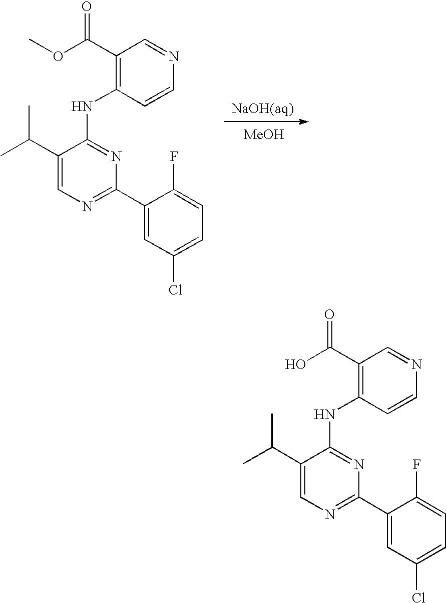

The ester (630 mg, 1.57 mmole) was suspended in 10 ml methanol and treated with 4 ml 2.0M NaOH(aq). The mixture was refluxed for 30 min, then cooled reaction mixture evaporated under vacuum to remove methanol. The aqueous solution was acidified with 6M HCl (pH 5), filtered to obtain product 180 mg.

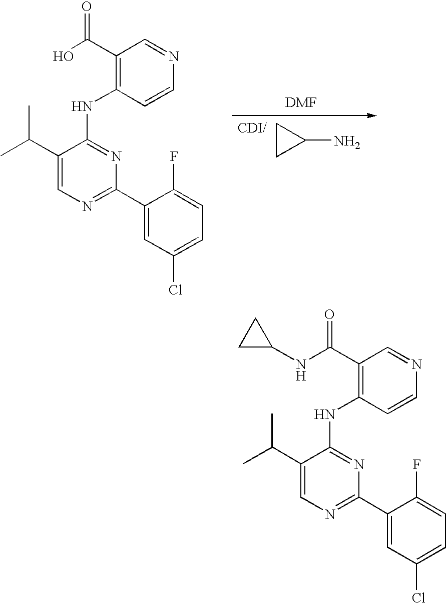

The acid (193 mg, 0.5 mmole) was suspended in DMF (anh. 6 ml) and treated with carbonyl dimimidazole (162 mg, 1.0 mmole) and heated to 60° C. for 2 hours. Cyclopropylamine (114 mg, 2.0 mmole) was added and the solution stirred overnight at room temperature. Filtered mixture and filtrate subjected to HPLC purification. Isolated 34 mg product.

### EXAMPLE 8

### Preparation of Compound 122

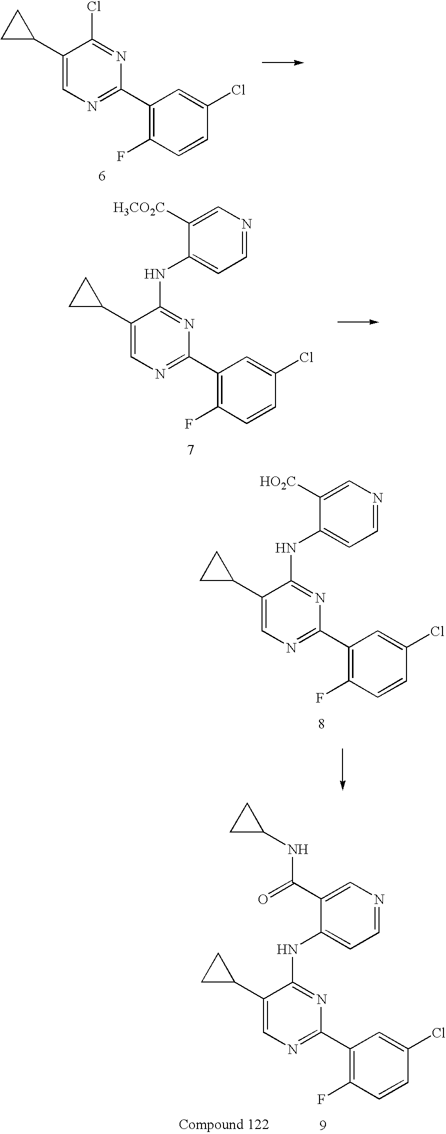

Preparation of 7:

To 1.42 g (5.0 mmol) of (6), was added 2.2 g (7.0 mmol) cesium carbonate, 0.056 g (0.25 mmol) Pd(Ac)2, .233 g (0.44 mmol) BINAP, and 0.912 g (6.0 mmol) of 4-amino-3-methylester pyridine. 10 ml of anhydrous 1-4-Dioxane was added and the mixture was heated to 90° C. overnight. Dioxane was removed by reduced pressure and material was washed with ethylacetate.

Preparation of 8:

To 0.35 g (1.24 mmol) of (7) was added 8 ml of methanol and 3 ml of a 1M NaOH solution. Mixture was heated to 70° C. for 2 hrs, cooled then acidified to pH5 using 1M HCl. Product was collected by vacuum filtration, washed with a small amount of water and dried in vacuum oven.

Preparation of 9:

To .223 g (0.589 mmol) of (8), was added 0.19 g (0.18 mmol) of N,N′-Carbonyldiimidazole. The mixture was treated with 4 ml of anhydrous DMF and heated to 70° C. for 2 hrs. Reaction was cooled to room temperature and 0.168 g (2.9 mmol) of cyclopropylamine was added and the reaction stirred at room temperature overnight. Reaction was then filtered and purified by prep HPLC.

### EXAMPLE 9

### Preparation of Compound 136

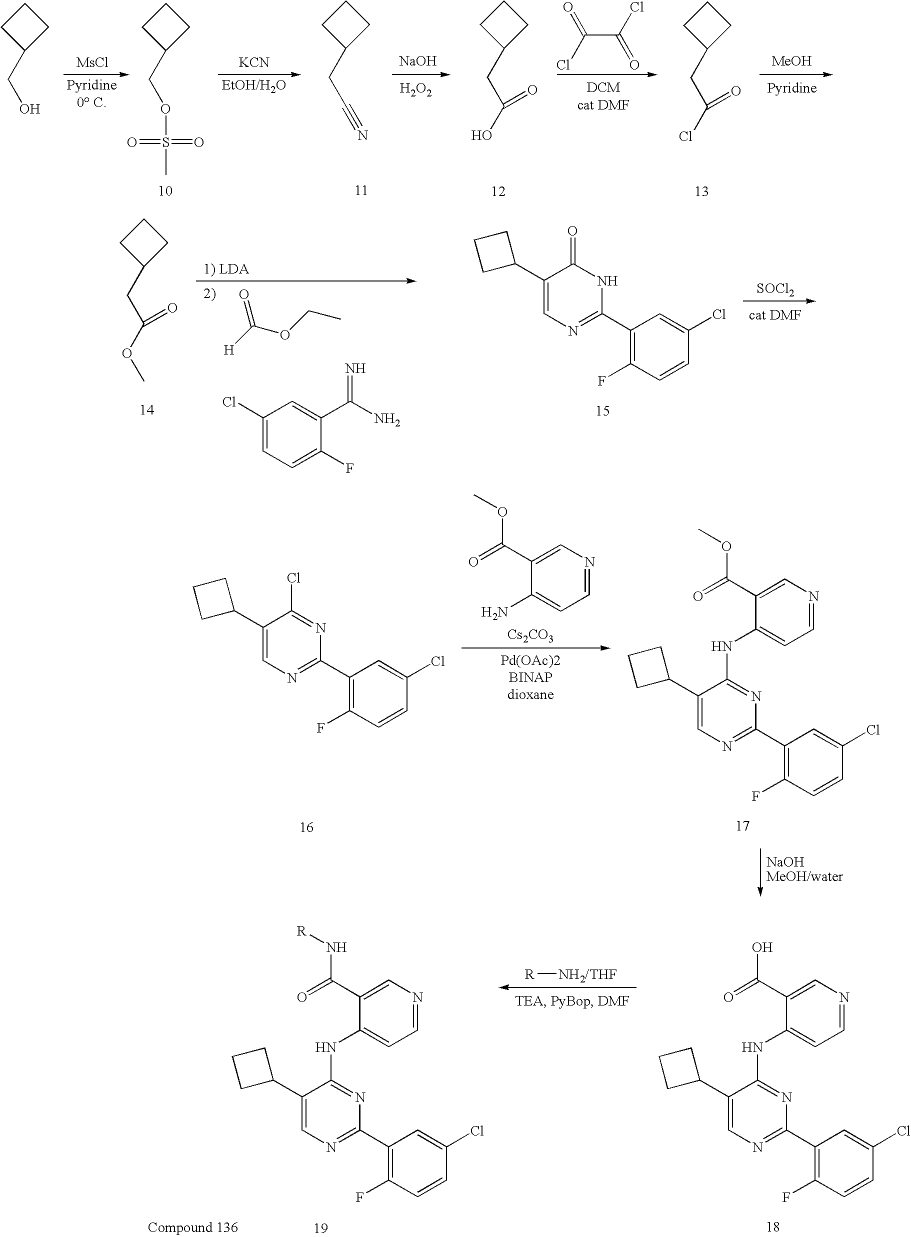

Preparation of 10:

A mixture of cyclobutylmethanol (25 g, 0.290 mole) and methanesulfonyl chloride (33.25 g, 0.290 mole) was stirred at 0° C. while pyridine was added drop wise over 2.5 hours. Reaction mixture was kept at 0° C. overnight, then combined with 150 ml ice cold 10% HCl. The mixture was extracted with diethyl ether (3×125 ml). Combined extracts were washed with water (2×20 ml) followed by saturated sodium bicarbonate (30 ml). Dried extract over anhydrous sodium sulfate and solvent removed under reduced pressure to give 35.58 g product.

Preparation of 11:

Cyclobutymethylmesylate (35.38 g 0.215 mole) was dissolved in 250 ml 80% ethanol/water and treated with potassium cyanide (25.25 g, 0.388, 1.8 eq) and reaction mixture refluxed overnight. Poured reaction mixture into 200 ml water and extracted with diethyl ether (2×100 ml), then washed with saturated sodium chloride (˜50 ml). Dried ether over sodium sulfate (anh.). The dark brown solution was passed over florisil (˜10 cm I.D. ×15 cm) twice to remove brown color. Removed solvent to give crude product, which was purified further by vacuum distillation to give 9.5 g product.

Preparation of 12:

An ice cooled bath of sodium hydroxide (40 g) in 50 ml water was stirred while a 30% hydrogen peroxide solution (50 ml) was added slowly maintaining cool temperature. Cyclobutylacetonitrile (9.5 g, 0.10 mole) was added slowly, solution stirred 30 min then heated to reflux for 2 days. Cooled reaction mixture, extracted with 50 ml chloroform to remove unreacted nitrile. Acidified aqueous layer with conc. HCl to pH 2, extracted cooled mixture with chloroform (3×150 ml). Dried chloroform extract over magnesium sulfate (anh.). Evaporated solvent to give 8.63 g product.

Preparation of 13:

Cyclobutylacetic acid (8.63 g, 75.6 mmole) was dissolved in dichloromethane containing 2 drops dimethylformamide and oxallyl chloride (45 ml, 2M dichloromethane) was added drop wise over 30 min at room temperature. The reaction mixture was stirred at room temperature for 3 hours, and then solvent removed to give 8.6 g product.

Preparation of 14:

Cyclobutyl acetyl chloride (8.6 g, 64.8 mmole) was added drop wise to a stirred solution of pyridine (10.48 ml, 129.6 mmole) in methanol (105 ml). The solution was stirred overnight at room temperature. Most of the excess methanol was removed under vacuum. Solution was poured onto 150 ml water, extracted with diethyl ether (3×125 ml). Combined extracts were washed with 25 ml 10% HCl, water (25 ml) and saturated sodium bicarbonate (25 ml), water (25 ml), saturated sodium chloride (25 ml). Ether was dried over anhydrous sodium sulfate and solvent removed to give 5.90 g product.

Preparation of 15:

To a solution of diisopropylamine (7.15 ml, 50.63 mmole) in 20 ml anhydrous tetrahydrofuran at −20° C., was added n-butyl lithium (2.5M hexanes, 22 ml, 55.23 mmole) drop wise. The solution was stirred at 0° C. for 40 min, cooled reaction mixture to −78° C. and methyl cyclobutyl acetate (5.9 g, 46.03 mmole) was added drop wise, the reaction mixture stirred at −78° C. for 30 min. Ethyl formate (3.71 ml, 46.03 mmole) was added and reaction mixture was warmed to −10° C. for 1 hour, then room temp 1 hour. 5-chloro-2-fluorobenzamidine (7.94 g, 46.03 mmole) was dissolved in 20 ml tetrahydrofuran and solution added to the reaction mixture drop wise over 10 min, then refluxed overnight. Removed most of tetrahydrofuran under vacuum, and residue taken up in 200 ml water. Washed aqueous solution with diethyl ether (2×75 ml) which removed dark color. Aqueous phase was acidified with glacial acetic acid to pH 5. Product precipitated from solution. Filtered solid, washed with water and vacuum dried to give 3.77 g product. (29% yield).

Preparation of 16:

2-(5-chloro-2-fluoro)-5-cyclobutylpyrimidine-4-one (3.75 g, 13.5 mmole) was suspended in thionyl chloride (15 ml, 205 mmole), added 2 drops dimethylformamide and heated mixture to 80° C. for 30 min. Starting material was completed dissolved at this time. Removed excess thionyl choride under vacuum and residue was poured onto ice water, extracted with chloroform, chloroform later washed with 10% sodium carbonate, dried over anhydrous. Sodium sulfate and solvent removed to give 3.98 g product. (99%)

Preparation of 17:

2-(5-chloro-2-fluoro)-4-chloro-5-cyclobutylpyrimidine (1.48 g, 5 mmole), cesium carbonate (2.28 g, 7 mmole), palladium(II) acetate (56.1 mg 0.25 mmole), BINAP (233 mg, 0.375 mmole) and methyl 4-aminopyridine-3-carboxylate (912 mg, 6 mmole) were combined in dioxane and heated to 80° C. overnight. Removed solvent under vacuum, triturated residue with ethyl acetate, filtered solid, washed with ethyl acetate to give 4.20 g solid, estimated to contain 1.92 g product, and remaining cesium carbonate. This material was used directly without further purification.

Preparation of 18:

VIII (4.20 g, estimated 1.92 g starting material+cesium carbonate) was suspended in methanol 10 ml, and 10 ml 1M sodium hydroxide. Refluxed solution 1 hour, then cooled mixture, removed methanol under vacuum, acidified aqueous solution to pH 4 with 1M HCl, filtered solid washing with water to give 1.30 g product after vacuum oven drying.

Preparation of 19:

IX (130 mg, 0.326 mmole) was suspended in dimethylformamide (8 ml). To this was added Pybop (254 mg, 0.489 mmole), triethylamine (49 microliters, 0.359 mmole) and 2M methyl amine/THF (815 microliters, 1.63 mmole) and reaction stirred at room temperature for 3 hours. The reaction mixture was filtered through 0.45 micron filter and subjected to HPLC purification to give 61 mg product.

### EXAMPLE 10

### Preparation of Compound 135

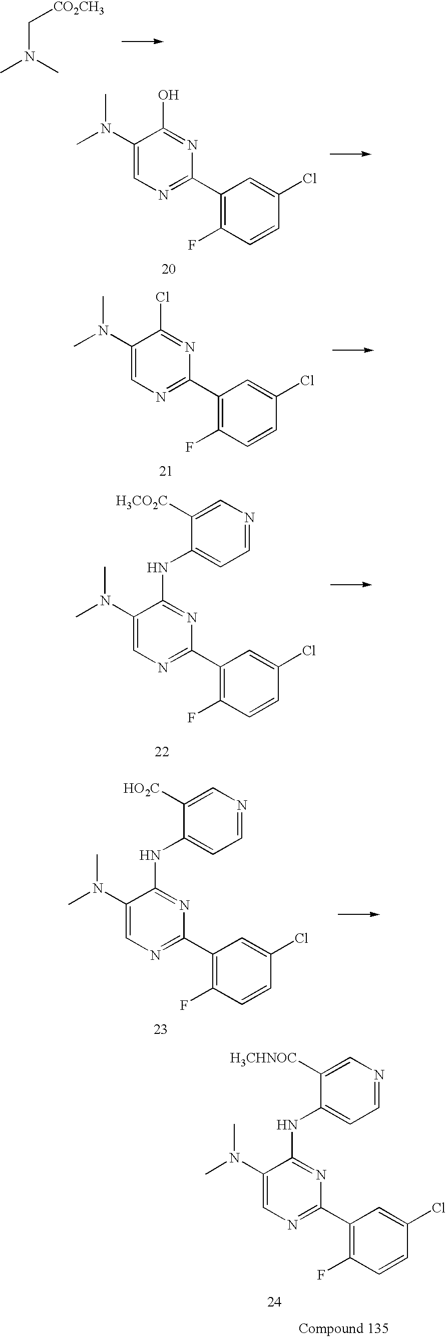

Preparation of 20:

Solid sodium metal pieces, (2.11 g, 92.0 mmol) was washed with hexane and crushed into smaller pieces. Hexane was removed and sodium pieces were added to a stirred solution at 0° C. of N,N-Dimethylglycine methyl ester, (10.78 g, 92.0 mmolin anhydrous ether (80 ml)). Ethylformate (7.4 ml, 92.0 mmol) was added dropwise to this solution and the reaction was stirred at room temperature for 3 hours. The rxn solution turns a creamy yellow consistency. To this mixture, 5-chloro-2-fluorobenzamidine, (15.9 g, 92.0 mmol) dissolved in 100 ml of 200 proof ethanol was syringe into reaction flask and the mixture was refluxed gently overnight. Solvent is then removed under reduced pressure and slurry is taken up into chloroform and extracted with water. Aqueous layer was adjusted to pH 7 and extracted with chloroform. Combined organic solvent was dried using magnesium sulfate and concentrated. Crude product is then washed with 20% ethylacetate/Hexane. Yield is 4.3 g, 17.5%.

Preparation of 21:

2-(5-chloro-2-fluorobenzyl)-5-cyclopropyl-pyrimidone, (0.46 g, 1.61 mmol) was treated with (2 ml, 15.7 mmol) of phosphorus oxychloride and refluxed for 2 hrs. Solvent was removed under reduced pressure and product was extracted into chloroform and washed with a saturated solution of sodiumhydrogen carbonate with ice. Organic solvent was dried using magnesium sulfate and concentrated. Reaction produced 0.43 g of product, 95% yield.

Preparation of 22:

Imino chloride (21), (0.43 g, 1.5 mmol) was dissolved in 5 ml of anhydrous 1,4-dioxane. To this (0.29 g, 1.9 mmol) of 5, (0.018 g, 0.080 mmol) of palladium acetate, (0.075 g, 0.121 mmol) of BINAP, and (0.786 g, 2.41 mmol) of cesium carbonate was added at once. The reaction was refluxed for 3 hours, cooled and the dioxane was evaporated off. Crude product is washed with ethylacetate. Crude product is a mixture with cesium carbonate remaining. No yield was taken.

Preparation of 23:

To (22) was added 15 ml of methanol and 3 ml of a 1M NaOH solution. Mixture was heated to 70° C. for 2 hrs, cooled then acidified to pH4 using 1M HCl. Product was collected by vacuum filtration, washed with a small amount of water and dried in vacuum oven. Received 0.064 g, 10.3% collective yield from imino chloride (21).

Preparation of 24:

To (0.064 g, 0.166 mmol) of (23), was added (0.054 g, 0.330 mmol) of N,N′-Carbonyldiimidazole. The mixture was treated with 5 ml of anhydrous DMF and heated to 70° C. for 2 hrs. Reaction was cooled to room temperature and 0.249 ml (0.498 mmol) of methylamine was added and the reaction stirred at room temperature overnight. Reaction was then filtered and purified by prep HPLC. Received 0.0152 g of material, 22.7% yield.

### EXAMPLE 11

### Preparation of Compound 108

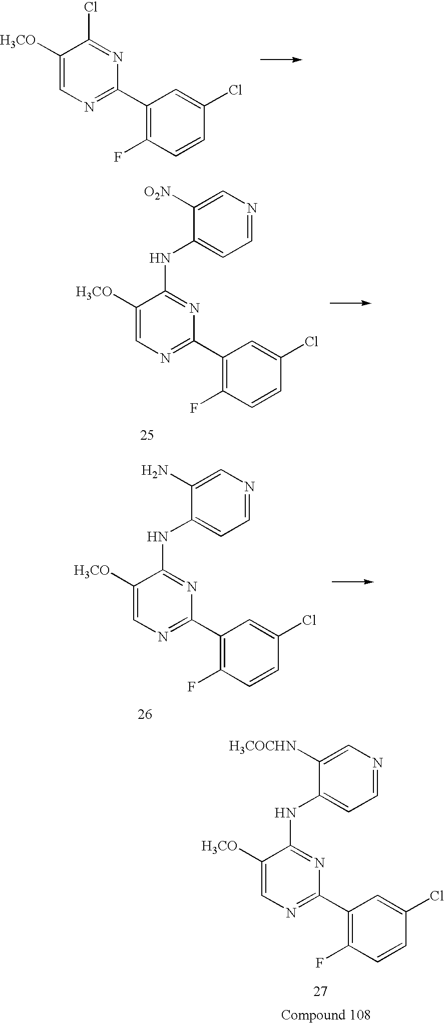

Preparation of 25:

To a solution of 4-amino-3-nitropyridine (300 mg, 2.15 mmol, 1 eq) in dry 1,4-dioxane (25 ml) were added Pd(OAC)₂ (24.1 mg, 0.107 mmol, 0.05 eq), BINAPP (100 mg, 0.162 mmol, 0.075 eq), 1054 mg of Cs₂CO₃ (3.23 mmol, 1.5 eq) followed by 704 mg of 2-fluoro-5-chlorobenzamidine 1. The reaction solution was stirred and heated at 90° C. under nitrogen protection for 3 days. The reaction mixture was cooled to room temperature and filtered through Celite®. The solvent was removed in vacuo to give a brown residue 25 (42% yield) which was purified by silica gel column chromatography eluted by (MeOH/DCM, 10/90).

Preparation of 26:

To a solution of 25 (400 mg, 1.06 mmol) in fresh methanol (20 ml) was added Pd/C (10% wt). The reaction system was evacuated and filled with hydrogen under 1 atm for 20 h. The catalyst was removed by filtration and the filtrate was evaporated to give crude 26 (39% yield). The crude product was purified by column chromatography on silica gel (MeOH/DCM, 5/95).

Preparation of 27:

To a solution of 26 (100 mg, 0.29 mmol, 1 eq) in fresh DCM (10 ml) was added 72 mg of 1-ethyl-3-(3-dimethylaminopropyl)-carbodimide (EDC) (72.2 mg, 0.377 mmol, 1.3 eq) followed by acetic acid (13.3 mg, 0.29 mmol, 1 eq). The reaction solution was stirred at room temperature for 8 hrs. The solvent was removed in vacuo to give 27 (99% yield).

### EXAMPLE 12

### Preparation of Compound 150

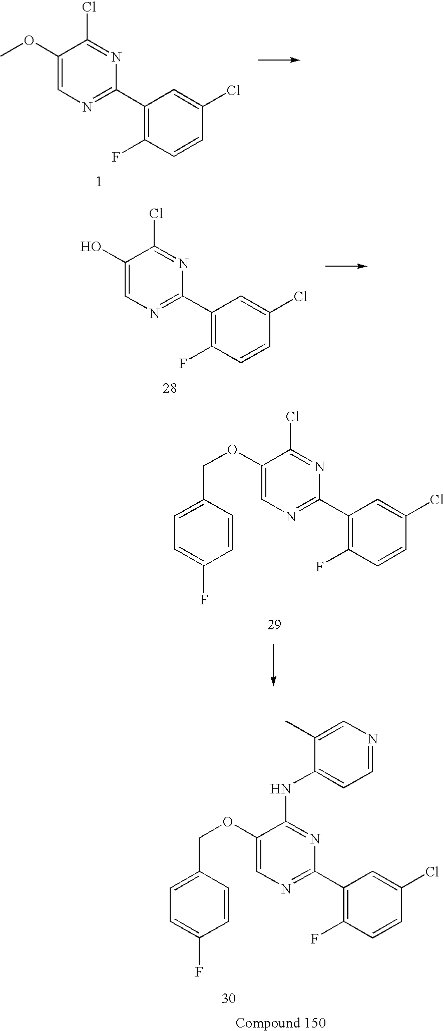

A suspension of 5-methoxy-4-chloropyrimidine 1 (853 mg, 1.8 mmol) and AlCl₃ (5 g, 21.9 mmol, 12 eq.) in methylene chloride (50 mL) was heated to reflux for 48 h, and then poured into a solution of 1N HCl (50 mL). The mixture was extracted with CH₂Cl₂ (5×100 mL) and the extracts dried over Na₂SO₄. Evaporation of the solvent followed by chromatography (0-10% MeOH in CH₂Cl₂) gave the product 28 as a white solid (772 mg, 95%).

A suspension of 5, hydroxy-4-chloropyrimidine 28 (50 mg, 0.19 mmol), 4, fluoro-benzyl bromide (56 mg, 0.29 mmol, 1.5 eq.) and K₂CO₃ (40 mg, 0.29 mmol, 1.5 eq.) in dry DMF (2 mL) was heated to 60° C. overnight followed by evaporation and chromatography (CH₂Cl₂) to give the product 29 as a cream solid (43 mg, 61% yield).

To a solution of the chloropyrimidine 29 (43 mg, 0.12 mmol) in dry dioxane (3 mL) was added successively Pd₂(dba)₃ (5 mg, 5 mol %), Rac-BINAP (6 mg, 7.5 mol %), 3, methyl-4-aminopyridine (15 mg, 0.14 mmol, 1.2 eq.) and NaO^{t}Bu (14 mg, 0.14 mmol, 1.2 eq.). The mixture was heated at 50° C. for 5 h then cooled and evaporated. The crude residue was purified by HPLC to give the desired product 30, lyophilized as a TFA salt (7.4 mg).

### EXAMPLE 13

### Preparation of Compound 159

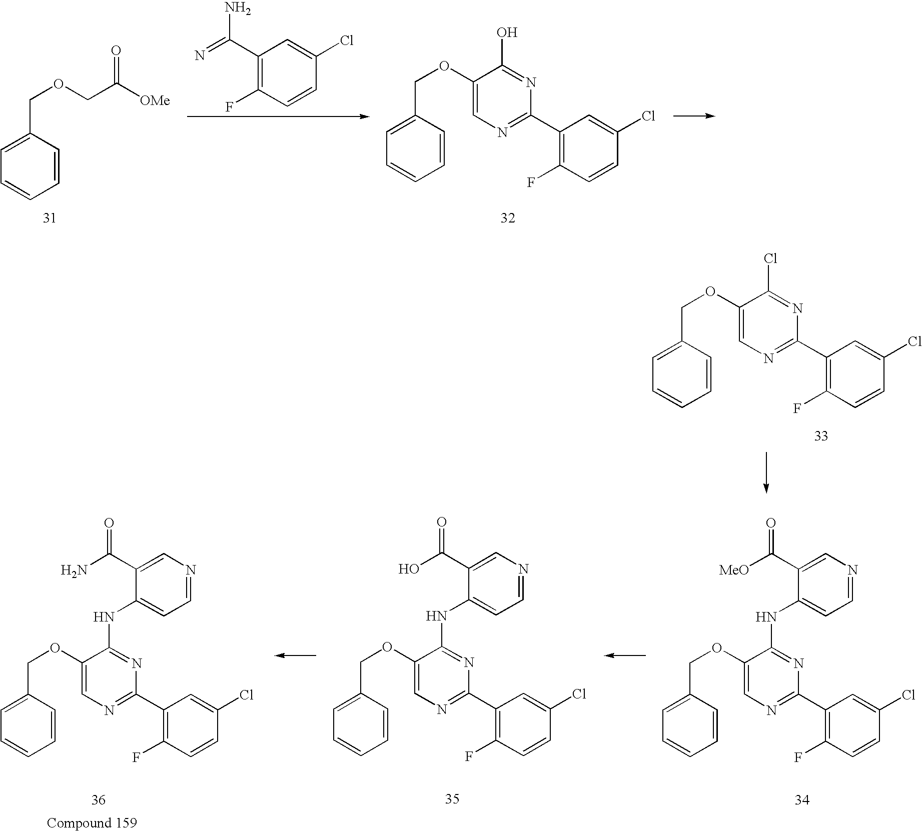

The 5-benzyloxy analogs were synthesized using the same conditions as those for the 5-methoxy analogs, but using methyl-benzyloxyacetate 31 as the starting material.

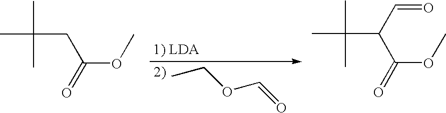

To a solution of diisopropylamine (20.58 g, 204 mmole) in 60 ml tetrahydrofuran (anh.) at −20° C. was added dropwise, n-butyllithium (2.5M hexane, 88 ml, 222 mmole). The solution was stirred at 0° C. for 40 min. The mixture was then cooled to −78° C. and methyl t-butyl acetate (24.1.0 g, 185 mmole) was added dropwise, the reaction mixture was stirred at −78° C. for 30 min. Ethyl formate (13.70 g, 185 mmole) was then added and the reaction mixture was warmed to room temperature with stirring for 18 hours. The reaction mixture was poured into 300 ml ice water. The organic layer was extracted with 1M sodium hydroxide (2×40 ml) and added to the aqueous layer. The aqueous layer was acidified with 40% sulfuric acid to pH 5.0 with cooling. The solution was extracted with diethyl ether (5×40 ml), combined ether extract washed with saturated sodium chloride, dried over sodium sulfate (anh.) and solvent removed to give product as a liquid (11.4 g, 39% yield). This material was used without further purification.

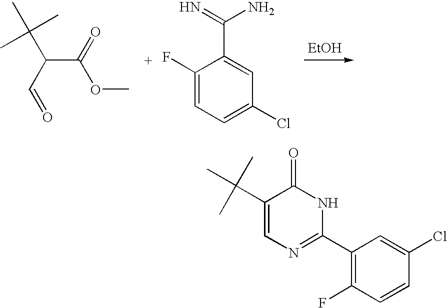

5-chloro-2-fluorobenzamidine (7.39 g, 42.8 mmole) and methyl 1-formyl-t-butyl acetate (6.78 g, 42.8 mmole) was dissolved in ethanol (75 ml) and heated to reflux for 2 hours. Removed ethanol by rotary evaporation, residue taken up in chloroform (300 ml), extracted with 1M sodium hydroxide (4×40 ml). Combined aqueous extract was acidified with 1M hydrochloric acid. Product was extracted with ethyl acetate (3×100 ml), combined extract dried over sodium sulfate (anh.) and solvent removed to give the product 2.02 g (17% yield).

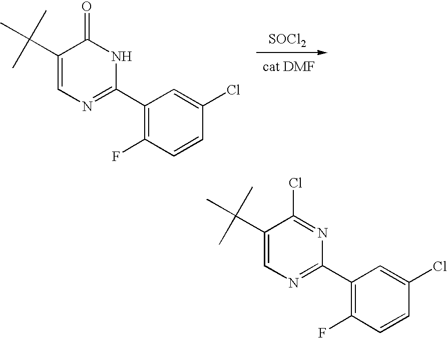

2-(5-chloro-2-fluorophenyl)-5-t-butylprimidine-4-one (2.02, 7.20 mmole) was suspended in thionyl chloride (10 ml) and 3 drops DMF were added. The mixture was heated to 80° C. for 30 min, removed excess thionyl chloride under vacuum. The residue was treated with ice (50 ml) and chloroform (50 ml). Extracted product into chloroform. Washed chloroform with 10% sodium carbonate (cold) and dried chloroform layer over sodium sulfate (anh,) Removed solvent to give 2.00 g product. (93% yield)

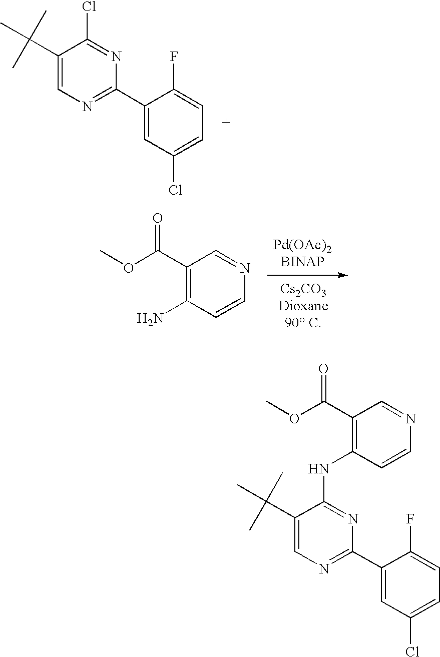

BINAP (311 mg, 0.50 mmole) and palladium(II)acetate (74 mg, 0.334 mmole were combined in 10 ml dioxane (anh) and heated for 5 min, followed by addition of 2-(5-chloro-2-fluorophenyl)-4-chloro-5-t-butyllpyrimidine (2.00 g, 6.68 mmole), methyl 4-amino-3-pyridinecarboxylate (1.22 g, 8.0 mmole) and cesium carbonate (3.05 g, 9.38 mmole). The mixture was heated to 90° C. overnight. Removed dioxane under vacuum, the solid residue was triturated with ethyl acetate (20 ml) and filtered to give 3.1 5 g product which contains cesium carbonate and was used directly in next step without further purification.

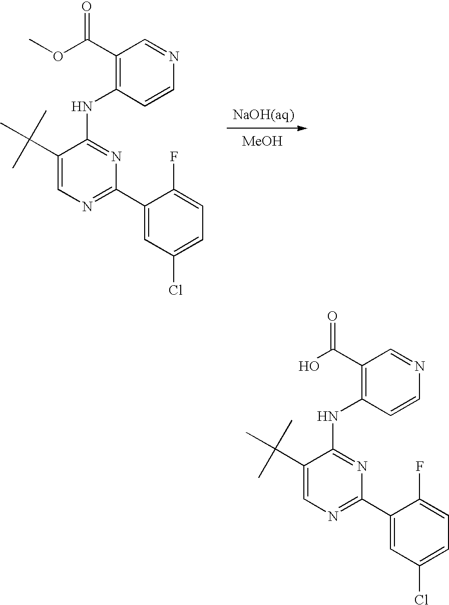

The ester (3.15 g,) was suspended in 10 ml methanol and treated with 4 ml 2.0 M NaOH(aq). The mixture was refluxed for 1 hour, then cooled reaction mixture evaporated under vacuum to remove methanol. The aqueous solution was acidified with 6M HCl (pH 5), filtered to obtain product 2.25 g.

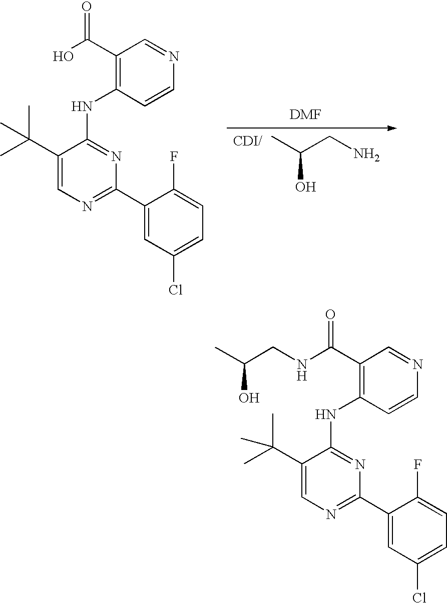

The acid (100 mg, 0.25 mmole) was suspended in DMF (anh. 3 ml) and treated with carbonyl dimimidazole (81 mg, 0.5 mmole) and heated to 60° C. for 2 hours. S(+)-1-amino-2-propanol (75 mg, 1.0 mmole) was added and the solution stirred overnight at room temperature. Filtered mixture and filtrate subjected to HPLC purification. Isolated 12 mg product.

### EXAMPLE 14

### Activity of the Invention Compounds

The compounds of the invention are tested for their ability to inhibit TGFβ by a TGFβ R¹ autophosphorylation protocol. This was conducted as follows: Compound dilutions and reagents were prepared fresh daily. Compounds were diluted from DMSO stock solutions to 2 times the desired assay concentration, keeping final DMSO concentration in the assay less than or equal to 1%. TGFβ R1 was diluted to 4 times the desired assay concentration in buffer+DTT. ATP was diluted into 4× reaction buffer, and gamma-³³P-ATP was added at 60 uCi/mL.

The assay was performed by adding 10 ul of the enzyme to 20 ul of the compound solution. The reaction was initiated by the addition of 10 ul of ATP mix. Final assay conditions included 10 uM ATP, 170 nM TGFβ R1, and 1M DTT in 20 mM MOPS, pH7. The reactions were incubated at room temperature for 20 minutes. The reactions were stopped by transferring 23 ul of reaction mixture onto a phosphocellulose 96-well filter plate, which had been pre-wetted with 15 ul of 0.25M H₃PO₄ per well. After 5 minutes, the wells were washed 4× with 75 mM H₃PO₄ and once with 95% ethanol. The plate was dried, scintillation cocktail was added to each well, and the wells were counted in a Packard TopCount microplate scintillation counter.

The illustrated compounds provide, in this assay, IC₅₀ values in the range of 0.05-50 micromolar.

TABLE 1

| COM- | |
|---|---|
| POUND # | STRUCTURE |
| 1 | 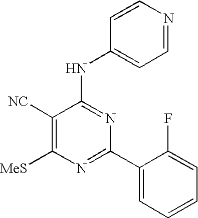 |
| 2 | 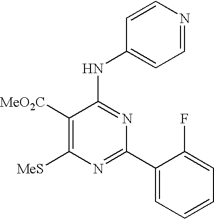 |
| 3 | 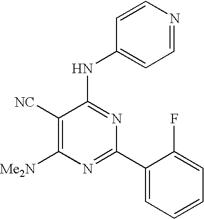 |
| 4 | 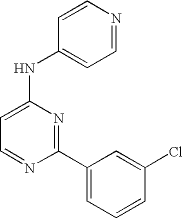 |
| 5 | 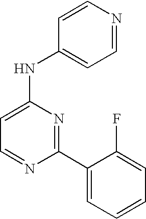 |
| 6 | 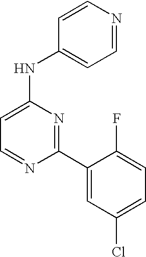 |
| 7 | 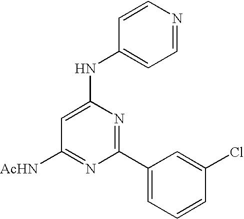 |
| 8 | 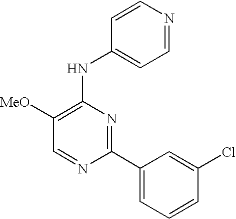 |
| 9 | 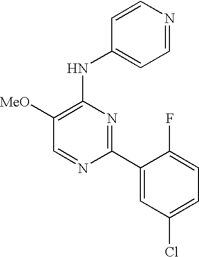 |
| 10 | 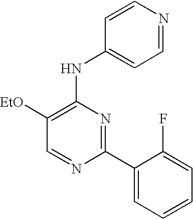 |
| 11 | 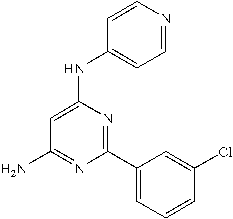 |
| 12 | 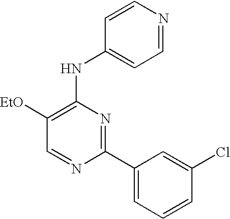 |
| 13 | 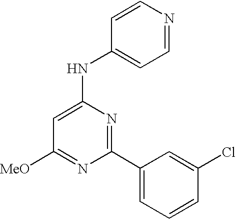 |
| 14 | 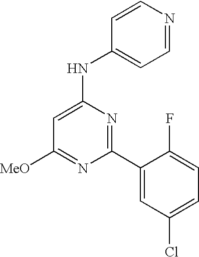 |
| 15 | 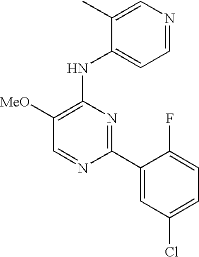 |
| 16 | 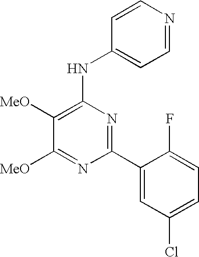 |
| 17 | 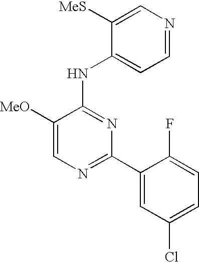 |
| 18 | 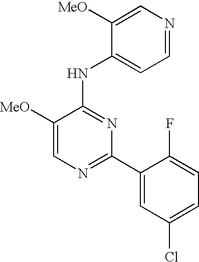 |
| 19 | 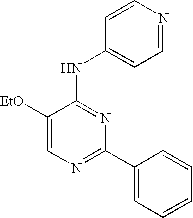 |
| 20 | 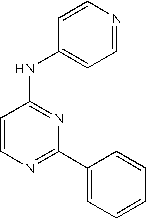 |
| 21 | 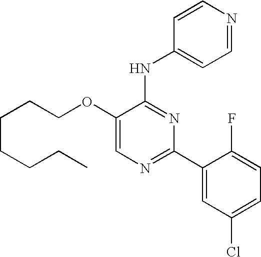 |
| 22 | 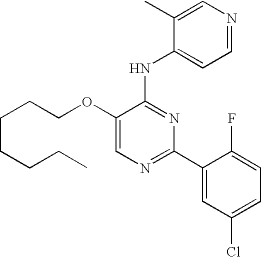 |
| 23 | 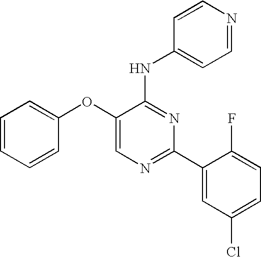 |
| 24 | 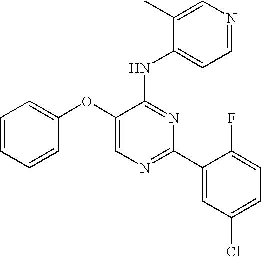 |
| 25 | 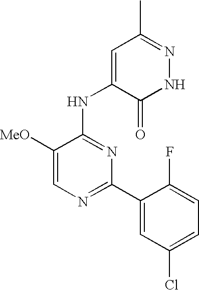 |
| 26 | 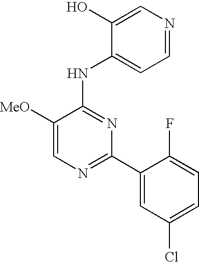 |
| 27 | 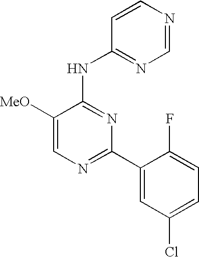 |
| 28 | 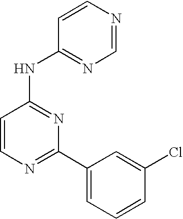 |
| 29 | 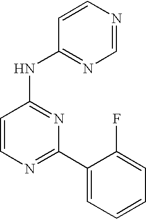 |
| 30 | 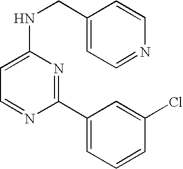 |
| 31 | 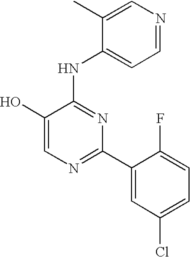 |
| 32 | 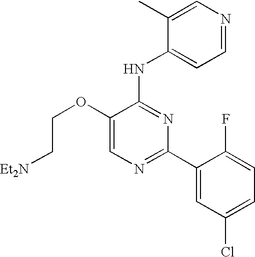 |
| 33 | 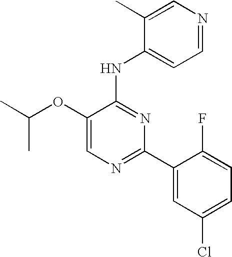 |
| 34 | 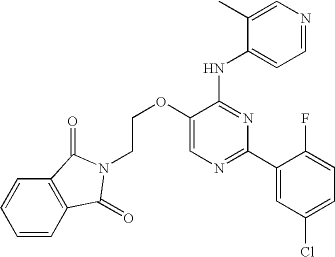 |
| 35 | 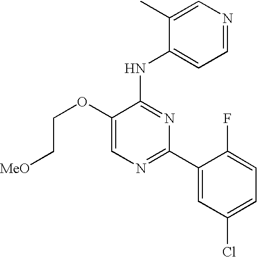 |
| 36 | 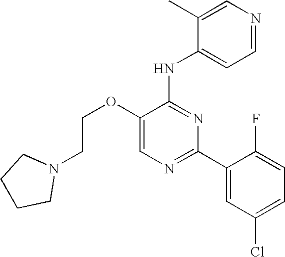 |
| 37 | 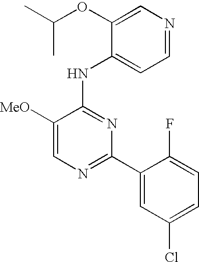 |
| 38 | 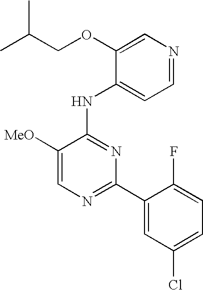 |
| 39 | 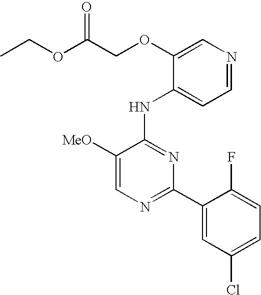 |
| 40 | 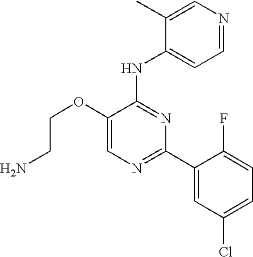 |
| 41 | 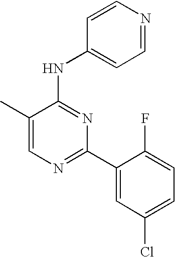 |
| 42 | 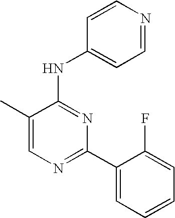 |
| 43 | 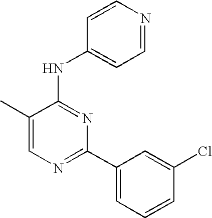 |
| 44 | 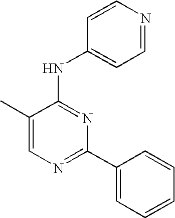 |
| 45 | 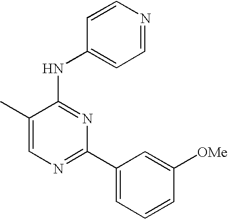 |
| 46 | 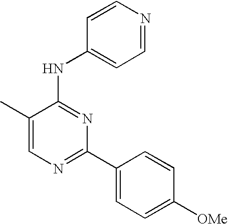 |
| 47 | 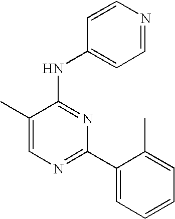 |
| 48 | 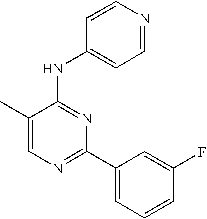 |
| 49 | 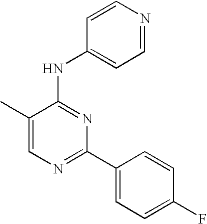 |
| 50 | 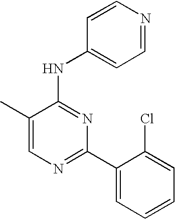 |
| 51 | 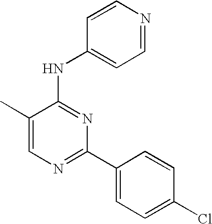 |
| 52 | 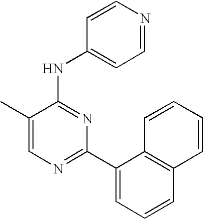 |
| 53 | 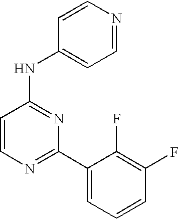 |
| 54 | 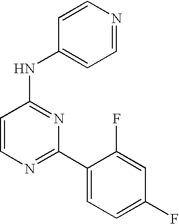 |
| 55 | 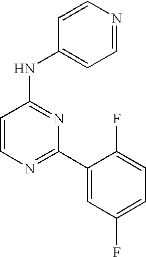 |
| 56 | 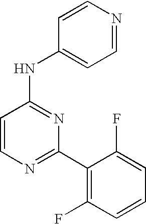 |
| 57 | 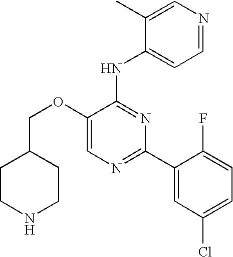 |
| 58 | 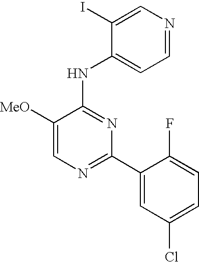 |
| 59 | 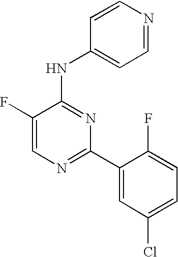 |
| 60 | 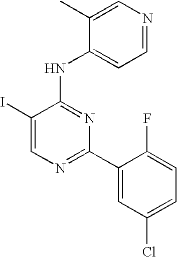 |
| 61 | 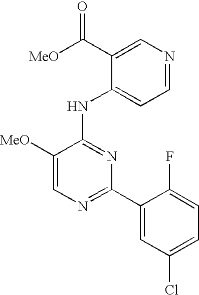 |
| 62 | 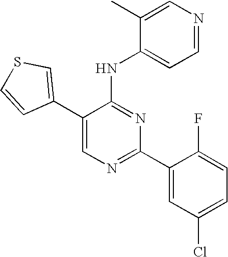 |
| 63 | 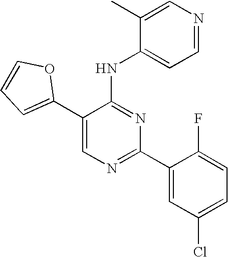 |
| 64 | 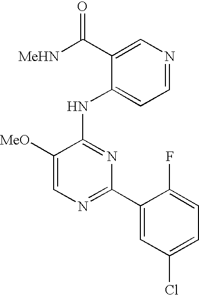 |
| 65 | 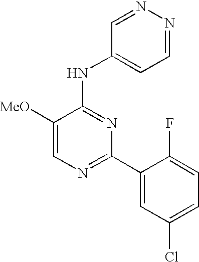 |
| 66 | 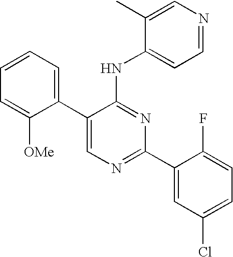 |
| 67 | 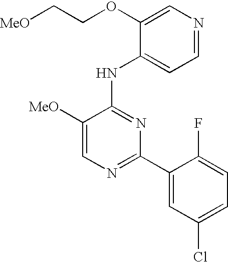 |
| 68 | 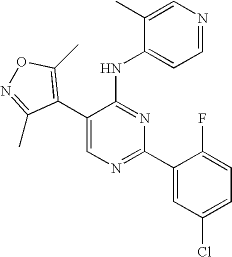 |
| 69 | 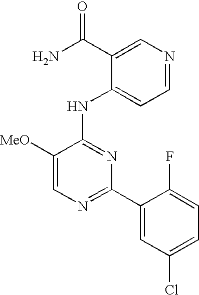 |
| 70 | 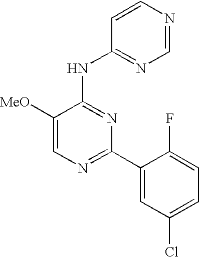 |
| 71 | 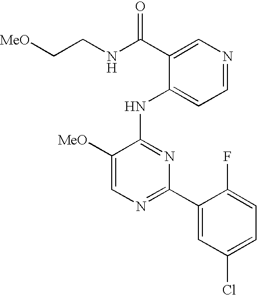 |
| 72 | 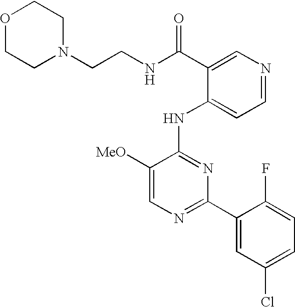 |
| 73 | 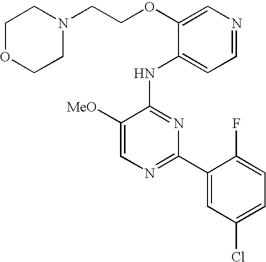 |
| 74 | 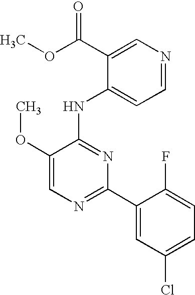 |
| 75 | 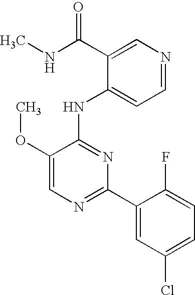 |
| 76 | 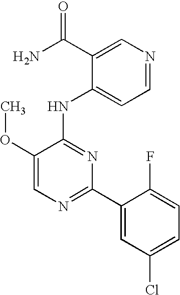 |
| 77 | 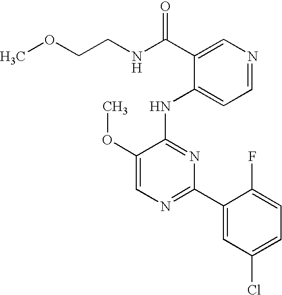 |
| 78 | 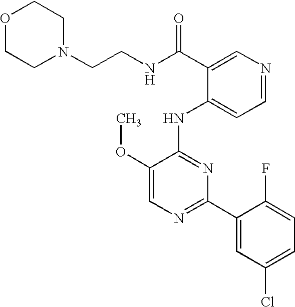 |
| 79 | 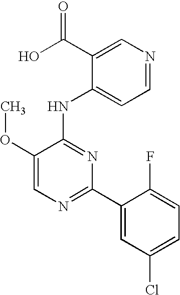 |
| 80 | 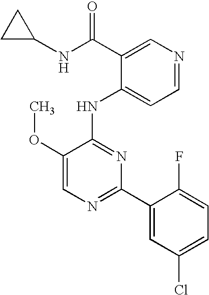 |
| 81 | 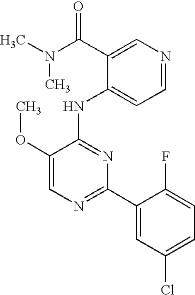 |
| 82 | 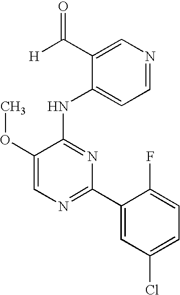 |
| 83 | 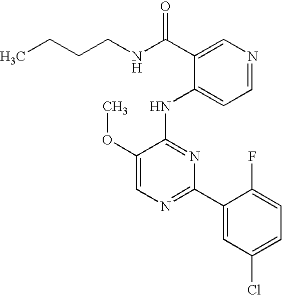 |
| 84 | 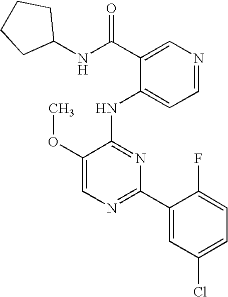 |
| 85 | 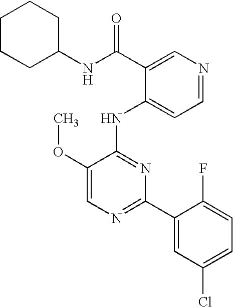 |
| 86 | 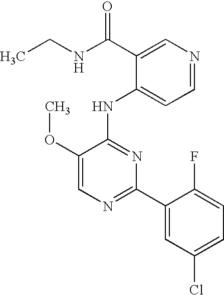 |
| 87 | 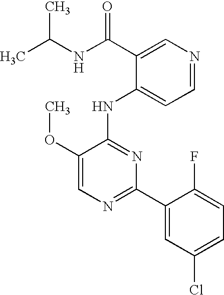 |
| 88 | 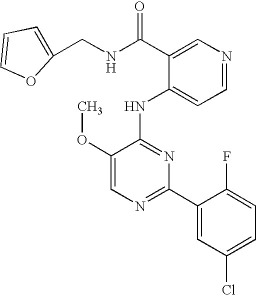 |
| 89 | 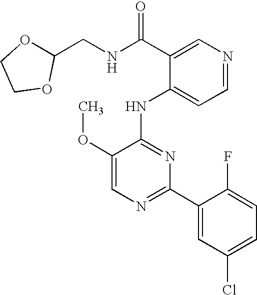 |
| 90 | 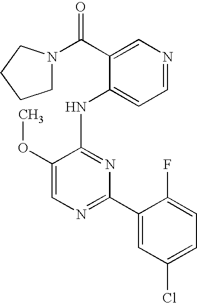 |
| 91 | 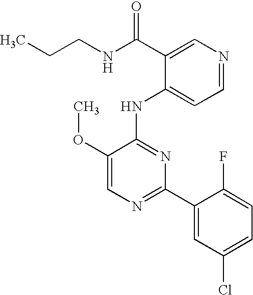 |
| 92 | 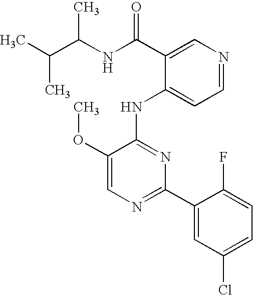 |
| 93 | 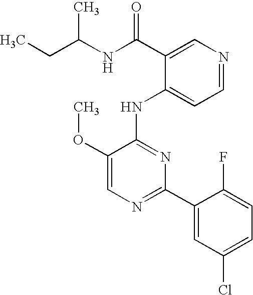 |
| 94 | 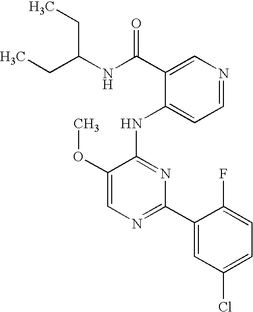 |
| 95 | 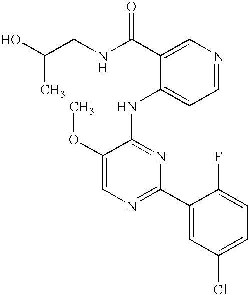 |
| 96 | 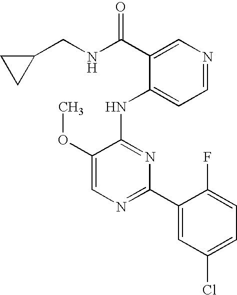 |
| 97 | 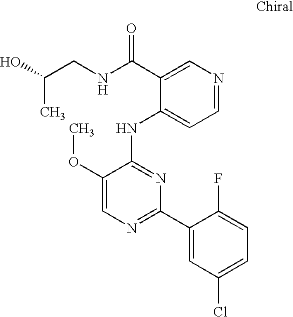 |
| 98 | 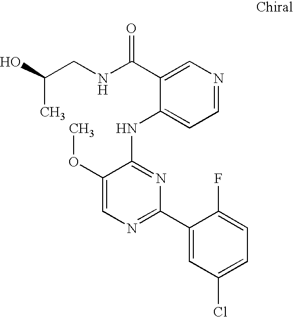 |
| 99 | 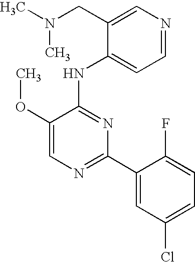 |
| 100 | 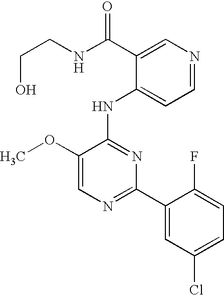 |
| 101 | 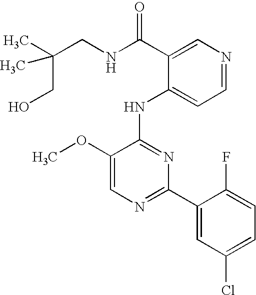 |
| 102 | 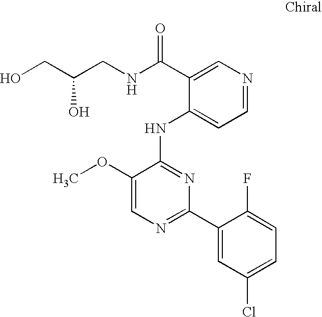 |
| 103 | 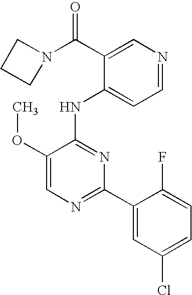 |
| 104 | 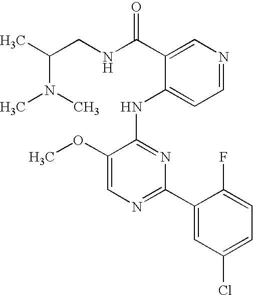 |
| 105 | 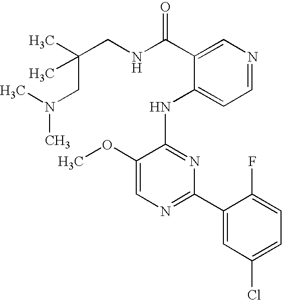 |
| 106 | 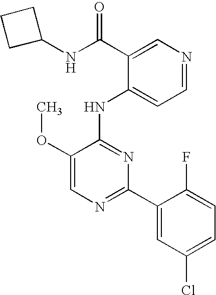 |
| 107 | 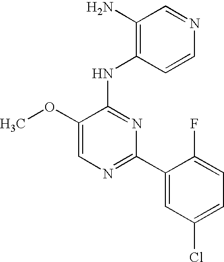 |
| 108 | 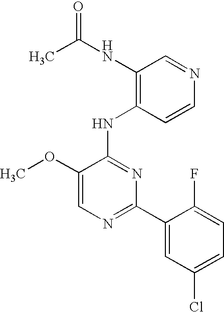 |
| 109 | 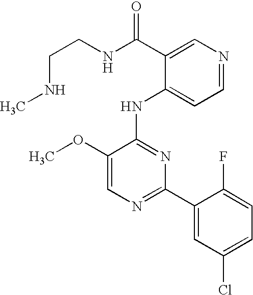 |
| 110 | 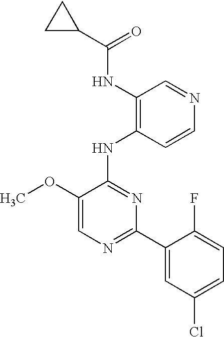 |
| 111 | 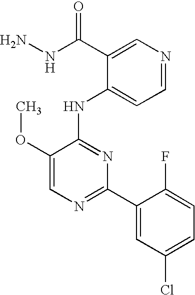 |
| 112 | 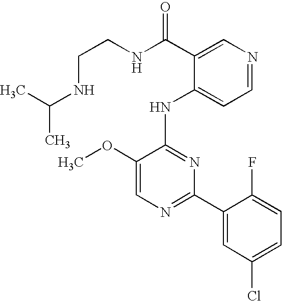 |
| 113 | 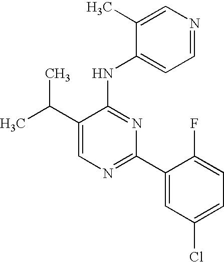 |
| 114 | 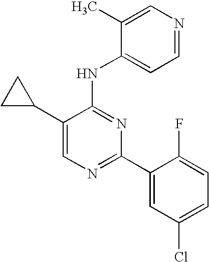 |
| 115 | 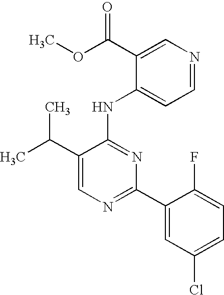 |
| 116 | 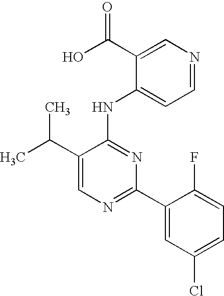 |
| 117 | 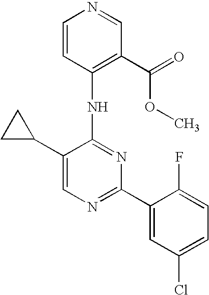 |
| 118 | 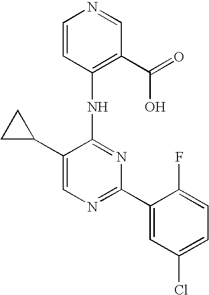 |
| 119 | 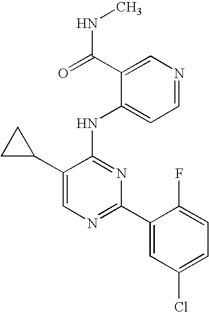 |
| 120 | 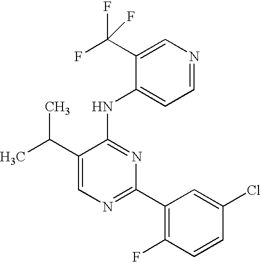 |
| 121 | 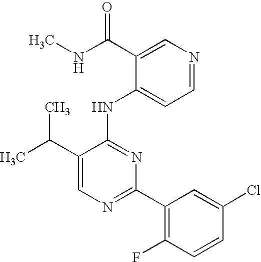 |
| 122 | 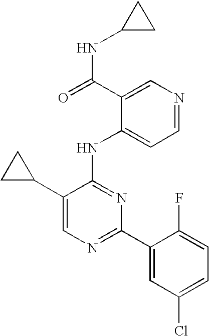 |
| 123 | 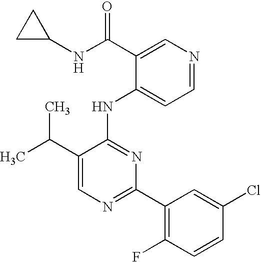 |
| 124 | 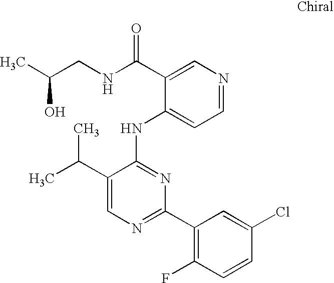 |
| 125 | 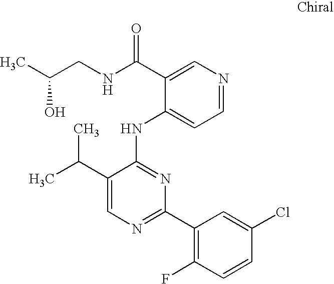 |
| 126 | 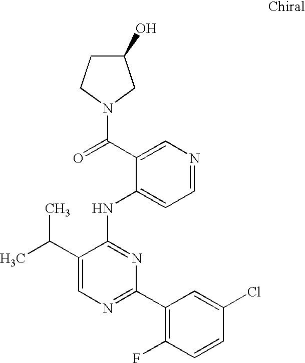 |
| 127 | 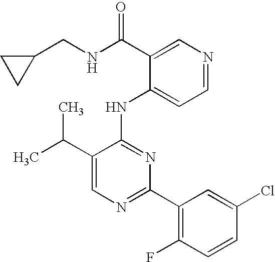 |
| 128 | 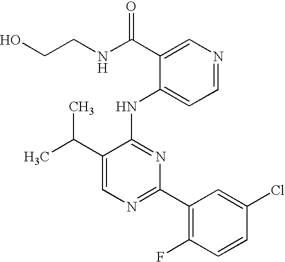 |
| 129 | 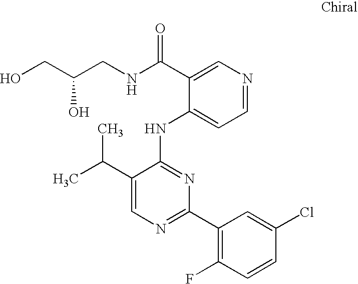 |
| 130 | 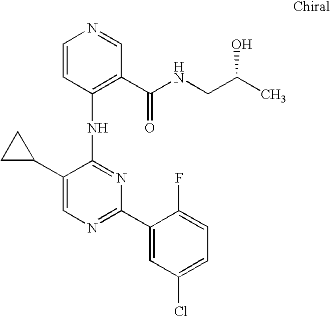 |
| 131 | 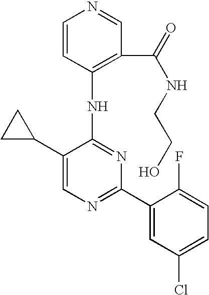 |
| 132 | 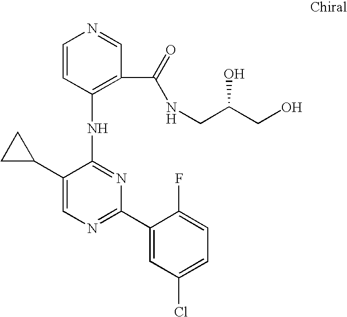 |
| 133 | 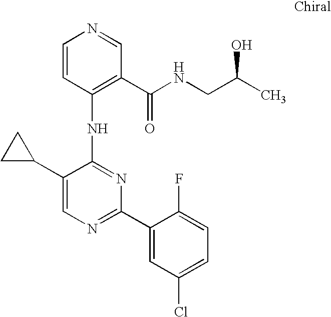 |
| 134 | 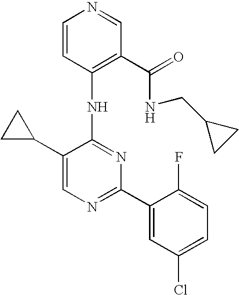 |
| 135 | 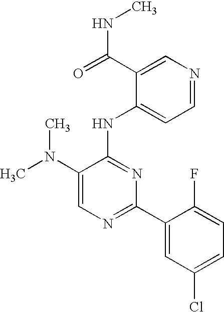 |
| 136 | 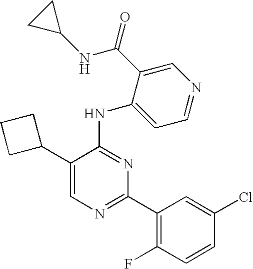 |
| 137 | 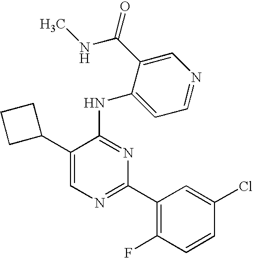 |
| 138 | 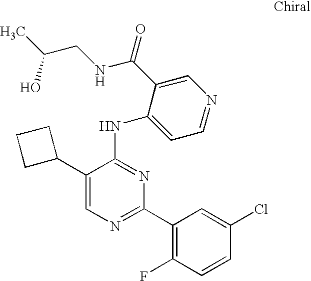 |
| 139 | 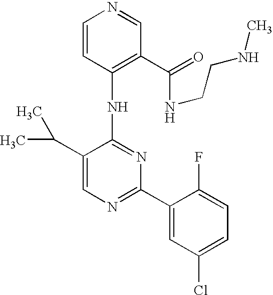 |
| 140 | 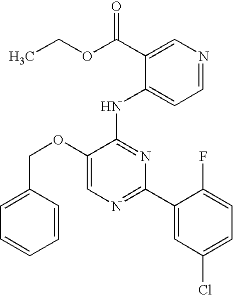 |
| 141 | 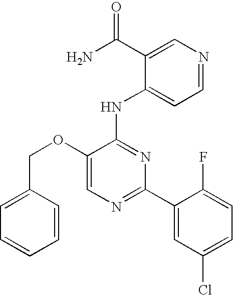 |
| 142 | 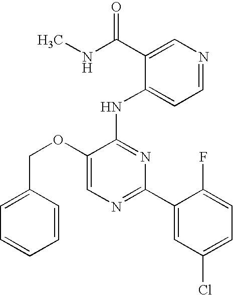 |
| 143 | 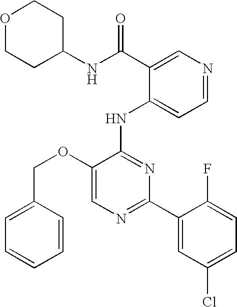 |
| 144 | 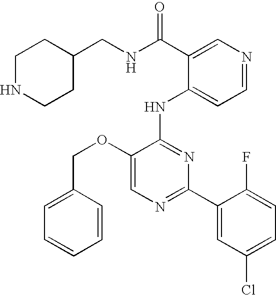 |
| 145 | 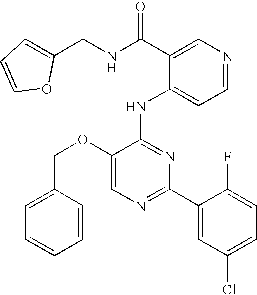 |
| 146 | 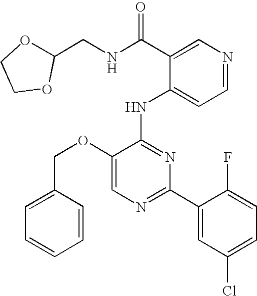 |
| 147 | 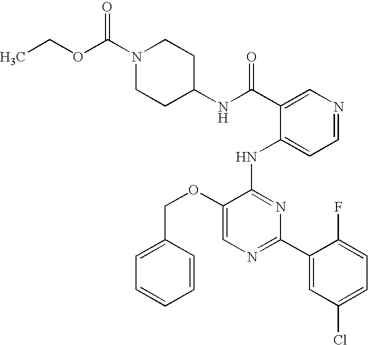 |
| 148 | 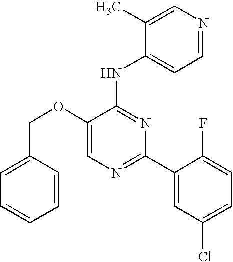 |
| 149 | 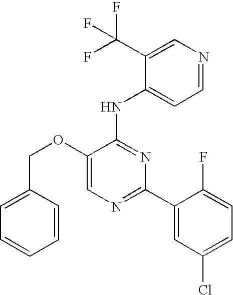 |
| 150 | 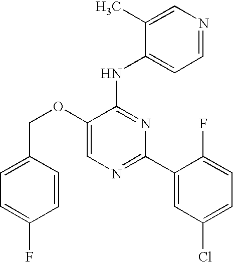 |
| 151 | 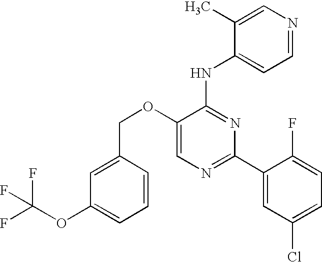 |
| 152 | 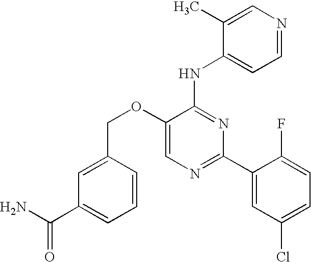 |
| 153 | 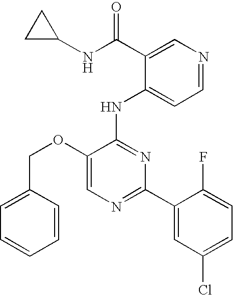 |
| 154 | 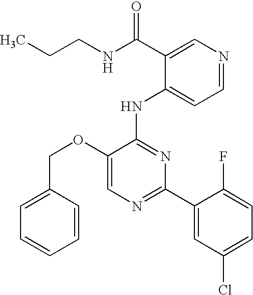 |
| 155 | 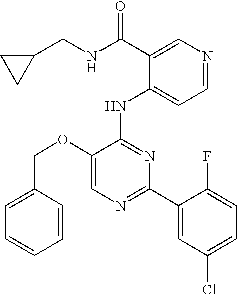 |
| 156 | 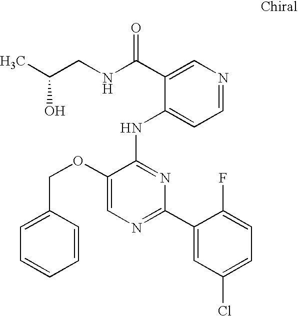 |
| 157 | 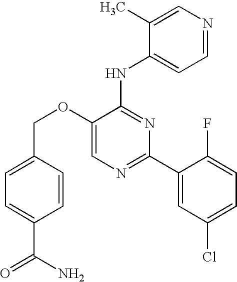 |
| 158 | 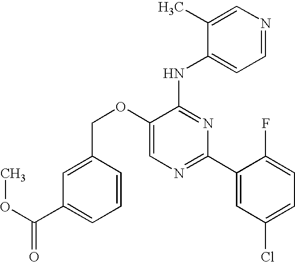 |
| 159 | 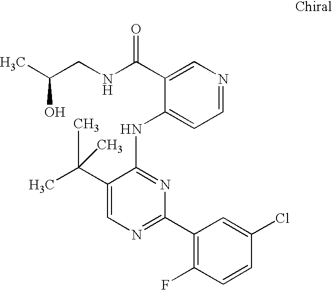 |
| 160 | 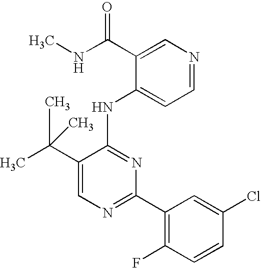 |

1. A compound of the formula 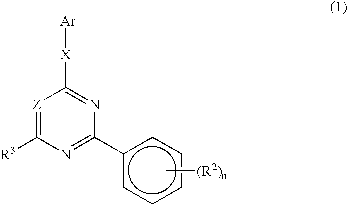
and the pharmaceutically acceptable salts and prodrug forms thereof; wherein
Ar represents an optionally substituted aromatic or optionally substituted heteroaromatic moiety containing 5-12 ring members wherein said heteroaromatic moiety contains one or more O, S, and/or N, with a proviso that optionally substituted Ar is not 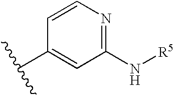
wherein R⁵ is H, alkyl (1-6C), alkenyl (2-6C), alkynyl (2-6C), an aromatic or heteroaromatic moiety containing 5-11 ring members;
X is NR¹, O, or S;
R¹is H, alkyl (1-8C), alkenyl (2-8C), or alkynyl (2-8C);
Z represents N or CR⁴;
each of R³ and R⁴ is independently H, or a non-interfering substituent;
each R² is independently a non-interfering substituent; and
n is 0-5:

2. The compound of claim 1 wherein each R³ and R⁴ is independently H, alkyl, alkenyl, alkynyl, acyl, aryl, alkylaryl, aroyl, O-aryl, O-alkylaryl, O-aroyl, NR-aryl, NR-alkylaryl, NR-aroyl, or the hetero forms of any of the foregoing, halo, OR, NR₂, SR, —SOR, —NRSOR, —NRSO₂R, —SO₂R, —OCOR, —NRCOR, —NRCONR₂, —NRCOOR, —OCONR₂, —COOR, —SO₃R, —CONR₂, —SO₂NR₂, —CN, —CF₃, or —NO₂, wherein each R is independently H or alkyl (1-10C);
wherein any alkyl, alkenyl, alkynyl, acyl or aryl groups contained in R³ and/or R⁴ may contain one or more heteroatoms and/or optionally be further substituted.

3. The compound of claim 1 wherein each R² is independently alkyl, alkenyl, alkynylacyl, aryl, alkylaryl, aroyl, O-aryl, O-alkylaryl, O-aroyl, NR-aryl!, NR-alkylaryl, NR-aroyl, or the hetero forms of any of the foregoing, halo, OR, NR₂, SR, —SOR, —NRSOR, —NRSO₂R, —SO₂R, —OCOR, —NRCOR, —NRCONR₂, —NRCOOR, —OCONR₂, —COOR, —SO₃R, —CONR₂, —SO₂NR₂, —CN, —CF₃, or —NO₂, wherein each R is independently H or lower alkyl (1-4C), wherein any alkyl, alkenyl, alkynyl, acyl or aryl groups contained in R² may contain one or more heteroatoms and/or may optionally be further substituted.

4. The compound of claim 1, wherein the substituents on the aromatic moiety of Ar are selected from the group consisting of alkyl, alkenyl, alkynyl, acyl, aryl, alkylaryl, aroyl, O-aryl, O-alkylaryl, O-aroyl, NR-aryl, NR-alkylaryl, NR-aroyl, or the hetero forms of any of the foregoing, halo, OR, NR₂, SR, —SOR, —NRSOR, —NRSO₂R, —SO₂R, —OCOR, —NRCOR, —NRCONR₂, —NRCOOR, —OCONR₂, —COOR, —SO₃R, —CONR₂, —SO₂NR₂, —CN, —CF₃, and —NO₂, wherein each R is independently H or alkyl (1-10C), and wherein any alkyl, alkenyl, alkynyl, acyl or aryl moieties contained in the substituent may contain one or more heteroatoms and/or may further be substituted by the foregoing substituents.

5. The compound of claim 1, wherein Ar is optionally substituted phenyl, 2-, 3- or 4-pyridyl, indolyl, 2- or 4-pyrimidyl, or benzimidazolyl.

6. The compound of claim 1, wherein n is 0-3.

7. The compound of claim 1, wherein R¹ is H or lower alkyl (1-4C).

8. The compound of claim 2, wherein each R³ and R⁴ is independently H, alkyl (1-10C), OR, SR or NR₂ wherein R is H or alkyl (1-10C), each optionally substituted.

9. The compound of claim 8, wherein said optional substituent is an aromatic moiety or a heterocyclic moiety, each optionally substituted.

10. The compound of claim 9, wherein at least one of R³ and R⁴ is H.

11. The compound of claim 3, wherein each R² is independently alkyl, alkoxy, or halo.

12. The compound of claim 11, wherein each R² is independently halo.

13. The compound of claim 4, wherein the substituents on the aromatic moiety of Ar are selected from the group consisting of alkyl, O-aryl, O-alkylaryl, NR-aryl, and N-alkylaryl wherein any alkyl or aryl contained in said substituent may further optionally be substituted.

14. The compound of claim 13, wherein said aryl includes 0, 1 or 2 substituents.

15. The compound of claim 14, wherein said aryl includes 0 or 1 substituents.

16. The compound of claim 2, wherein each R³ and R⁴ is independently H, CN, COOR, OR, SR, NR₂, alkyl (1-6C), acyl (1-6C), aryl, aryloxy, arylalkyloxy, wherein R is H or alkyl (1-10C) and wherein any alkyl or aryl portions of said substituents may further be substituted with the foregoing.

17. The compound of claim 1, wherein R¹ is H.

18. The compound of claim 5, wherein Ar is optionally substituted phenyl, 4-pyridyl, 3-pyridyl, 4-pyrimidyl, or 2-pyrimidyl.

19. The compound of claim 18, wherein Ar is 4-pyridyl.

20. A method to treat conditions associated with unwanted activity of TGFβ which method comprises administering to a subject in need of such treatment an effective amount of the compound of claim 1 or a pharmaceutical composition thereof.

21. A pharmaceutical composition which comprises the compound of formula (1) in admixture with at least one pharmaceutically acceptable excipient.
